(19) United States
(12) Patent Application Publication
(10) Pub. No.: US 2025/0262223 A1
(43) Pub. Date: Aug. 21, 2025

(54) METHODS FOR THE TREATMENT OF BIOLOGICAL AGING

(71) Applicant: BIOVIE INC., Carson City, NV (US)

(72) Inventors: Clarence Nathaniel Ahlem, San Diego, CA (US); Christopher L. Reading, San Diego, CA (US)

(21) Appl. No.: 19/190,210

(22) Filed: Apr. 25, 2025

Related U.S. Application Data
(63) Continuation of application No. PCT/US2023/035707, filed on Oct. 23, 2023.
(60) Provisional application No. 63/508,856, filed on Jun. 16, 2023.
(60) Provisional application No. 63/381,521, filed on Oct. 28, 2022.

(51) Int. Cl. A61K 31/566; A61P 25/28

(52) U.S. Cl. CPC A61K 31/566; A61P 25/28

(57) ABSTRACT

Disclosed herein are compositions and methods useful for the treatment or amelioration of various diseases, disorders, or conditions. Some aspects pertain to a pharmaceutical composition comprising 17-ethynyl-10R, 13S dimethyl 2, 3, 4, 7, 8R, 9S, 10, 11, 12, 13, 14S, 15, 16, 17-hexadecahydro-1H-cyclopenta[a]phenanthrene-3R, 7R, 17S-triol, including solid states thereof. Also presented herein is the surprising discovery that exposing a subject to the compositions disclosed herein can treat, reduce, or ameliorate a condition related to biological clocks.

### CROSS REFERENCE TO RELATED APPLICATIONS

This application is a continuation of PCT/US2023/035707, filed Oct. 23, 2023 and claims the benefit of priority to U.S. Provisional Application No. 63/381,521, filed Oct. 28, 2022, and U.S. Provisional Application No. 63/508,856 filed Jun. 16, 2023. All of the foregoing applications are incorporated herein by reference in their entireties for all purposes.

### FIELD OF THE DISCLOSURE

The present disclosure relates to the field of chemistry and medicine. More particularly, the present disclosure relates generally to methods for the treatment of biological aging using 17-ethynyl-10R, 13S-dimethyl 2, 3, 4, 7, 8R, 95, 10, 11, 12, 13, 145, 15, 16, 17-hexadecahydro-1H-cyclopenta[a]phenanthrene-3R, 7R, 17S-triol.

### BACKGROUND

While aging may be a complex multifactorial process with no single cause or treatment, the issue of whether aging can be classified as a disease is widely debated. Many strategies for extending organismal life spans have been proposed, including replacing cells and organs, comprehensive strategies for repairing the accumulated damage, using hormones to activate endogenous repair processes, and modulating the aging processes through specific mutations, gene therapy, and small molecule drugs. An animal's survival strongly depends on its ability to maintain homeostasis, achieved partly through intracellular and intercellular communication within and among different tissues.

Many biomarkers of aging have been proposed, including telomere length, intracellular and extracellular aggregates, racemization of the amino acids, and genetic instability. Gene expression and DNA methylation profiles change during aging, which may also be used as aging biomarkers. DNA methylation algorithms are increasingly used to estimate biological aging; however, how these proposed measures of whole-organism biological aging relate to aging in the brain is not known or well understood.

### SUMMARY OF THE DISCLOSURE

Aspects of the disclosure relate to a method to treat, reduce, or ameliorate a disease or condition associated with biological clocks in a subject in need thereof. In some embodiments, the method includes administering to the subject 17α-ethynylandrost-5-ene-3β,7β,17β-triol. In some embodiments, the disease or condition associated with biological clocks in the subject in need thereof is based on modulation of DNA methylation of genes associated with biological clocks. In some embodiments, the disease or condition associated with a biological clock in the subject in need thereof is associated with genes or genomic regions hypermethylated with age. In some embodiments, the disease or condition associated with a biological clock in the subject in need thereof is associated with genes or genomic regions hypomethylated with age. In some embodiments, the disease or condition associated with a biological clock in a subject in need thereof is associated with Tau phosphorylation. In some embodiments, the disease or condition associated with a biological clock in a subject in need thereof is associated with hyperglycemia. In some embodiments, the disease or condition associated with a biological clock in a subject in need thereof is associated with insulin resistance. In some embodiments, the disease or condition associate with biological clock is mild cognitive impairment or late onset Alzheimer's disease. In some embodiments, administering to the subject 17α-ethynylandrost-5-ene-3β,7β,17β-triol and at least one pharmaceutically acceptable excipient decreases a subject's Alzheimer's Disease Composite Score. In some embodiments, the subject experiences a decrease between about a 5% to about a 100% reduction in symptoms connected to Alzheimer's Disease Composite Score after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases pTau in the subject. In some embodiments, the subject experiences a decrease between about a 5% to about a 100% reduction in conditions or symptoms connected to pTau after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient increases leptin in the subject. In some embodiments, the subject experiences a decrease between about a 5% to about a 100% reduction in conditions or symptoms connected to leptin after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases DNA methylation in the subject. In some embodiments, the subject experiences a decrease between about a 5% to about a 100% reduction in conditions or symptoms connected to DNA methylation after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases cardiovascular risk in the subject. In some embodiments, the subject experiences a decrease between about a 5% to about a 100% reduction in conditions or symptoms connected to cardiovascular risk after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases DNA methylation age in the subject. In some embodiments, the subject experiences a decrease between about a 5% to about a 100% reduction in conditions or symptoms connected to DNA methylation after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases DNA methylation phenoage in the subject. In some embodiments, the subject experiences a decrease between about a 5% to about a 100% reduction in conditions or symptoms connected to DNA methylation phenoage after administration of 17α-ethynylandrost-5-ene-3β,7β,17β-triol and at least one pharmaceutically acceptable excipient. In some embodiments, administering to the subject 17α-ethynylandrost-5-ene-3β,7β,17β-triol and at least one pharmaceutically acceptable excipient decreases DNA methylation skin blood clock in the subject. In some embodiments, the subject experiences a decrease between about a 5% to about a 100% reduction in conditions or symptoms connected to DNA methylation skin blood clock after administration of 17α-ethynylandrost-5-ene-3β,7β,17β-triol and at least one pharmaceutically acceptable excipient. In some embodiments, the 17α-ethynylandrost-5-ene-3β,7β,17β-triol is administered orally. In some embodiments, the 17α-ethynylandrost-5-ene-3β,7β,17β-triol is administered intravenously. In some embodiments, the subject has a waist to hip ratio greater than or equal to approximately 0.90. In some embodiments, the subject has a waist to hip ratio greater than or equal to approximately 0.95. In some embodiments, the 17α-ethynylandrost-5-ene-3β,7β,17β-triol is a solid state form of 17α-ethynylandrost-5-ene-3β,7β,17β-triol. In some embodiments, the solid state form of 17α-ethynylandrost-5-ene-3β,7β,17β-triol is crystalline solvate of 17α-ethynylandrost-5-ene-3β,7β,17β-triol. In some embodiments, the crystalline solvate is crystalline methanolate 17α-ethynylandrost-5-ene-3β,7β,17β-triol. In some embodiments, the crystalline solvate is crystalline ethanolate 17α-ethynylandrost-5-ene-3β,7β,17β-triol. In some embodiments, the crystalline solvate is crystalline hydrate 17α-ethynylandrost-5-ene-3β,7β,17β-triol. In some embodiments, the crystalline solvate is Form III 17α-ethynylandrost-5-ene-3β,7β,17β-triol. In some embodiments, the crystalline solvate is Form IV 17α-ethynylandrost-5-ene-3β,7β,17β-triol. In some embodiments, the crystalline solvate is Form V 17α-ethynylandrost-5-ene-3β,7β,17β-triol. In some embodiments, the solid state form of 17α-ethynylandrost-5-ene-3β,7β,17β-triol is amorphous 17α-ethynylandrost-5-ene-3β,7β,17β-triol. In some embodiments, the pharmaceutical composition contains less than about 3% by weight of impurities.

Some embodiments relate to an in vitro screening method to identify a potential drug candidate or compound capable of treating, preventing, reducing, or ameliorating a disorder or disease. In some embodiments, the method includes (i) providing a sample for stimulation selected from the group consisting of a cell, tissue, blood, monocytes, microglia, macrophages, adipocytes, neuroblastoma, pheochromocytoma, and Lund human mesencephalic (LUHMES) cells, (ii) stimulating the sample with an agonist to induce a phenotype or phenotypic reaction, wherein the phenotype or phenotypic reaction substantially corresponds to a disease or condition associated with at least one DNA methylation at a CpG site in a region of DNA, (iii) contacting the one or more cells exhibiting the phenotype or phenotypic reaction with one or more potential drug candidate or compounds, (iv) determining a responsive change in the phenotype of the sample, and (v) providing the drug candidate or compound to a subject in need thereof to treat, reduce, prevent, or ameliorate a disease or condition associated with the DNA methylation in the subject. In some embodiments, the at least one DNA methylation at the CpG site is selected from the group consisting of AC073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof. In some embodiments, the phenotype or phenotypic reaction is selected from the group consisting of TNFα, GRC, CDR, MOCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab, or a combination thereof. In some embodiments, the phenotype or phenotypic reaction is selected from the group consisting of ADAS-Cog11, ADCOMS, CDR, CSF glucose, CSF pTau/Ab, frontal lobe volume, subcortical grey matter thickness, GRC, Heart rate, M oCA, PDQ-9, precuneus glutathione, QDRS, QDRS-behavior, QDRS-cognition, Systolic BP, Tau, TNFα, and weight. In some embodiments, the DNA methylation change is a decrease of >50%. In some embodiments, the DNA methylation change is a decrease of >55%. In some embodiments, the DNA methylation change is a decrease of >60%. In some embodiments, the phenotype or phenotypic reaction is decreased in TNF, CDR, QRDS-cognition, wherein the subject QDRS-cognition is improved. In some embodiments, the responsive change is a decrease or loss in the phenotype and the decrease or loss is indicative that the potential drug candidate or compound is capable of preventing, reducing, or ameliorating a neurodegenerative disorder or disease. In some embodiments, the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism. In some embodiments, the neurodegenerative disorder or disease is Parkinson's disease. In some embodiments, the neurodegenerative disorder is Alzheimer's disease. In some embodiments, the CpGs sites are interconnected with genes associated with Alzheimer's disease and related dementias. In some embodiments, the disease or condition associated with inflammatory TNF signaling. In some embodiments, the disease or condition associated with DNA methylation associated with Tau phosphorylation. In some embodiments, the disease or condition associated with DNA methylation associated with hyperglycemia. In some embodiments, the disease or condition associated with DNA methylation associated with insulin resistance. In some embodiments, the disease or condition associated with DNA methylation associated with obesity, ADAS-Cog11, ADCOMS, CDR, CSF glucose, CSF pTau/Aβ, frontal lobe volume, subcortical grey matter thickness, GRC, heart rate, M oCA, PDQ-9, precuneus glutathione, QDRS, QDRS-behavior, QDRS-cognition, systolic blood pressure, Tau, TNFα, and weight.

Some embodiments relate to a method of diagnose a patient with a disease or a condition. In some embodiments, the method includes (i) providing a patient with a potential drug candidate or compound capable of treating, preventing, reducing, or ameliorating a disorder or disease, (ii) identifying DNA methylation changes in the patient, and (iii) diagnosing the patient with a disease or condition associated with a biomarker associated with DNA methylation. In some embodiments, identifying DNA methylation changes in the patient identifies the CpGs decreased by more than 50%. In some embodiments, identifying DNA methylation changes is correlated with one or more clinical changes. In some embodiments, the DNA methylation changes at a CpG site is selected from the group consisting of AC073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof. In some embodiments, the disease or condition associated with a biomarker associated with DNA methylation is selected from the group consisting of TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Aβ, or a combination thereof. In some embodiments, the disease or condition associated with a biomarker associated with DNA methylation is selected from the group consisting of ADAS-Cog11, ADCOMS, CDR, CSF glucose, CSF pTau/Aβ, frontal lobe volume, subcortical grey matter thickness, GRC, Heart rate, MoCA, PDQ-9, precuneus glutathione, QDRS, QDRS-behavior, QDRS-cognition, Systolic BP, Tau, TNFα, and weight. In some embodiments, the biomarker associated with DNA methylation is decreased in TNF, CDR, QRDS-cognition, wherein the subject QDRS-cognition is improved. In some embodiments, the DNA methylation change is a decrease of >50%. In some embodiments, the DNA methylation change is a decrease of >55%. In some embodiments, the DNA methylation change is a decrease of >60%. In some embodiments, the disease or condition is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism. In some embodiments, the neurodegenerative disorder or disease is Parkinson's disease. In some embodiments, the neurodegenerative disorder is Alzheimer's disease. In some embodiments, the CpGs sites are interconnected with genes associated with Alzheimer's disease and related dementias. In some embodiments, the disease or condition associated with inflammatory TNF signaling. In some embodiments, the disease or condition associated with DNA methylation associated with Tau phosphorylation. In some embodiments, the disease or condition associated with DNA methylation associated with hyperglycemia. In some embodiments, the disease or condition associated with DNA methylation associated with insulin resistance. In some embodiments, the disease or condition associated with DNA methylation associated with obesity, ADAS-Cog11, ADCOMS, CDR, CSF glucose, CSF pTau/Aβ, frontal lobe volume, subcortical grey matter thickness, GRC, heart rate, MoCA, PDQ-9, precuneus glutathione, QDRS, QDRS-behavior, QDRS-cognition, systolic blood pressure, Tau, TNFα, and weight.

Not all objectives mentioned in this specification are necessarily achieved in all embodiments disclosed and/or claimed herein.

### BRIEF DESCRIPTION OF THE DRAWINGS

FIG. 1 illustrates a graph of Alzheimer's Disease Composite Score (ADCOMS) change.

FIG. 2 illustrates a graph of pTau v ADCOMS.

FIG. 3 illustrates a CSF pTau v ADCOMS for MMSE.

FIG. 4 illustrates a graph of DNA methylation scores (pre and post).

FIG. 5 illustrates a graph of DNA methylation scores for pack years.

FIG. 6 illustrates a graph estimating leptin scores.

FIG. 7 illustrates a graph measuring cardiovascular risk.

FIG. 8 illustrates a graph measuring DNA mAGE change.

FIG. 9 illustrates a graph measuring DNA PhenoAge change.

FIG. 10 illustrates a graph measuring DNAm change.

FIG. 11 illustrates a graph measuring DNAm changes following 14 weeks of bezisterim treatment.

FIG. 12 illustrates a chart representing a Phase 3, randomized, placebo-controlled trial of NE3107 (17α-ethynylandrost-5-ene-3β,7β,17β-triol) in subjects with mild to moderate probable Alzheimer's disease.

FIGS. 13A-13G illustrate graphs for improvements in the blinded assessments from the Phase 3, Randomized, Placebo-Controlled Trials.

FIGS. 14A-14B illustrates graphs for imaging sub-studies of vMRI hippocampus volume and amygdala volume in the blinded assessments from the Phase 3, Randomized, Placebo-Controlled Trials.

FIG. 15 illustrates graphs for imaging sub-studies FDG-PET in the blinded assessments from the Phase 3, Randomized, Placebo-Controlled Trials.

FIGS. 16A-16B illustrates graphs for neuropsychiatric inventory in the blinded assessments from the Phase 3, Randomized, Placebo-Controlled Trials.

FIGS. 17A-17C illustrates graphs representing increased fasting insulin and HOMA2-% B with decreased HOMA2-% S w/o hypoglycemia.

FIGS. 18A-18G illustrate graphs representing placebo effects in various assessments.

FIGS. 19-20 illustrate graphs representing ADAS-Cog12 spearman correlations.

FIGS. 21-22 illustrate graphs representing clinician rating of global change spearman correlations.

FIGS. 23-24 illustrate graphs representing mini-mental state exam spearman correlations.

FIG. 25 illustrate graphs representing ADCOMS spearman correlations.

FIG. 26 illustrate graphs representing CDR sum of boxes spearman correlations.

FIG. 27 illustrate graphs representing activities of daily living spearman correlations.

FIG. 28 illustrate graphs representing improvement in ADAS-Cog12 correlated with increased FDG-PET suvr.

FIG. 29 illustrate a graph representing an improvement in MMSE correlated with increased FDG-PET suvr.

FIG. 30 illustrate graphs representing an improvement in ADL correlated with increased FDG-PET SUVR.

FIG. 31 illustrate graphs representing an improvement in HOMA2 insulin sensitivity correlated with increased FDG-PET SUVR.

FIG. 32 illustrate graphs representing an improvement in cholesterol correlated with increased FDG-PET SUVR.

### DETAILED DESCRIPTION

The following description provides context and examples, but should not be interpreted to limit the scope of the disclosure covered by the claims that follow in this specification or in any other application that claims priority to this specification. No single component or collection of components is essential or indispensable. For example, in some embodiments one or more variables, such as Y or Y and Q may be omitted. Any feature, structure, component, material, step, or method that is described and/or illustrated in any embodiment in this specification can be used with or instead of any feature, structure, component, material, step, or method that is described and/or illustrated in any other embodiment in this specification.

### Definitions

As used herein and unless otherwise stated or implied by context, terms that are used herein have the meanings that are defined here. The descriptions of embodiments and examples that are described illustrate the disclosure and they are not intended to limit it in any way. Unless otherwise contraindicated or implied, e.g., by including mutually exclusive elements or options, in these definitions and throughout this specification, the terms “a” and “an” mean one or more and the term “or” means and/or.

A “formulation” or the like means a composition that one can administer to a subject, e.g., human or animal. Formulations are suitable for human or veterinary applications and would typically have expected characteristics for the formulation, e.g., parenteral formulations for human use would usually be sterile solutions or suspensions.

An “excipient”, “carrier”, “pharmaceutically acceptable carrier” or similar terms mean one or more component(s) or ingredient(s) that is acceptable in the sense of being compatible with the other ingredients in the disclosed compositions or formulations and not overly deleterious to the patient, animal, tissues or cells to which the formulation is to be administered.

“Effective amount” refers to the amount required to produce a desired effect (e.g., enhancing the half-life, bioavailability or efficacy of a compound described herein, treating biological aging in a subject, reducing DNA methylation in a subject, etc.

As used herein, “subject,” “host,” “patient,” and “individual” are used interchangeably and shall be given their ordinary meaning in the art and shall also refer to an organism that has cancer and/or leukemia. This includes mammals, e.g., a human, a non-human primate, ungulates, canines, felines, equines, mice, rats, and the like. The term “mammal” includes both human and non-human mammals.

“Preventing” in reference to a disease, disorder or condition refers to preventing a disease, disorder or condition, e.g., causing the clinical symptoms of the disease, disorder or condition not to develop. As used herein, the term “prevent,” “prevents,” or “prevention” (and grammatical equivalents thereof) may also refer to a delay in the onset of a disease or disorder or the lessening of symptoms upon onset of the disease or disorder. The terms are not meant to imply complete abolition of disease and encompass any type of prophylactic treatment that reduces the incidence of the condition or delays the onset and/or progression of the condition.

The terms “therapeutically effective amount” and “effective amount” refer to the amount of active pharmaceutical ingredient necessary to provide the desired pharmacologic result. In practice, the therapeutically effective amount will vary widely depending on the severity of the disease condition, age of the subject, and the desired therapeutic effect.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Methods of Treatment

Aspects of the present disclosure relate to methods to prevent, treat, reduce, or ameliorate a disease or condition associated with a biological clock in a subject in need thereof. In some embodiments, a method to prevent, treat, reduce, or ameliorate a disease or condition associated with a biological clock may include administering to a patient in need thereof an effective amount of a pharmaceutical composition. In some embodiments, the pharmaceutical composition includes a compound having the structure:

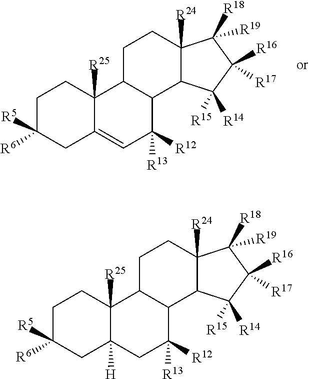

wherein, one of R⁵ and R⁶ is —OH and the other R⁵ and R⁶ is —H, one of R¹² and R¹³ is —OH and the other R¹² and R¹³ is —H, R¹⁴ and R¹⁵ are —H, R¹⁶ is —H, R¹⁷ is —H or —OH, R¹⁸ is —OH, R¹⁹ is ethynyl, and R²⁴ and R²⁵ are —CH₃.

In some embodiments, the pharmaceutical composition includes a compound having the structure

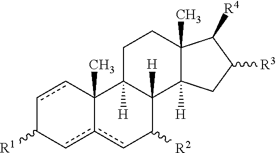

wherein, R¹ is —OH or an ester, R² is —OH or an ether, R³ is —OH, ═O, a halogen, an ester or ═CH₂, and R⁴ is an optionally substituted amine, an amide, an N-linked amino acid, ═NOH or —NHOH.

In some embodiments, the pharmaceutical composition includes an effective amount of a compound having the structure:

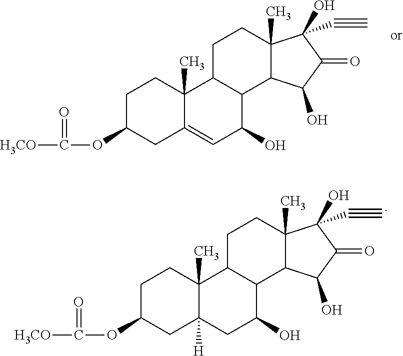 [SMILES: C#C[C@]1(O)C(=O)[C@@H](O)C2C3C(CC[C@@]21C)[C@@]1(C)CC[C@H](OC(=O)OC)CC1=C[C@@H]3O ; C#C[C@]1(O)C(=O)[C@@H](O)C2C3C(CC[C@@]21C)[C@@]1(C)CC[C@H](OC(=O)OC)C[C@@H]1C[C@@H]3O]

In some embodiments, the pharmaceutical composition includes an effective amount of a compound having the structure:

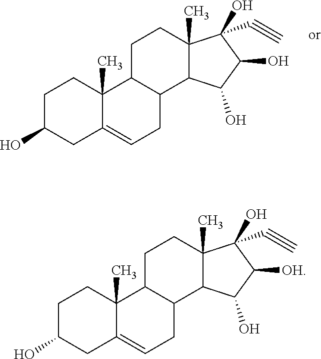 [SMILES: C#C[C@]1(O)[C@@H](O)[C@H](O)C2C3CC=C4C[C@@H](O)CC[C@]4(C)C3CC[C@@]21C ; C#C[C@]1(O)[C@@H](O)[C@H](O)C2C3CC=C4C[C@H](O)CC[C@]4(C)C3CC[C@@]21C]

In some embodiments, the pharmaceutical composition includes an effective amount of a compound having the structure:

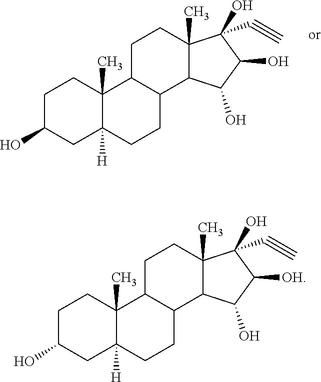 [SMILES: C#C[C@]1(O)[C@@H](O)[C@H](O)C2C3CC[C@H]4C[C@@H](O)CC[C@]4(C)C3CC[C@@]21C ; C#C[C@]1(O)[C@@H](O)[C@H](O)C2C3CC[C@H]4C[C@H](O)CC[C@]4(C)C3CC[C@@]21C]

In some embodiments, the pharmaceutical composition includes an effective amount of a compound having the structure:

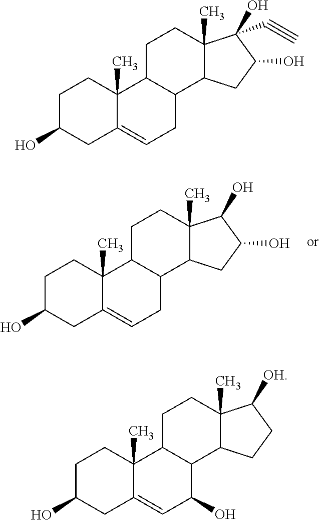 [SMILES: C#C[C@]1(O)[C@H](O)CC2C3CC=C4C[C@@H](O)CC[C@]4(C)C3CC[C@@]21C ; C[C@]12CC[C@H](O)CC1=CCC1C2CC[C@@]2(C)C1C[C@@H](O)[C@@H]2O ; C[C@]12CC[C@H](O)CC1=C[C@H](O)C1C2CC[C@@]2(C)C1CC[C@@H]2O]

In some embodiments, the pharmaceutical composition includes an effective amount of a compound having the structure:

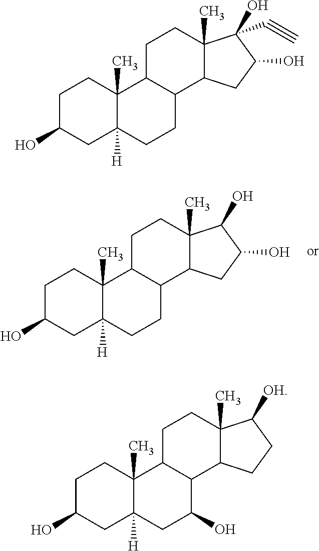 [SMILES: C#C[C@]1(O)[C@H](O)CC2C3CC[C@H]4C[C@@H](O)CC[C@]4(C)C3CC[C@@]21C ; C[C@]12CCC3C(CC[C@H]4C[C@@H](O)CC[C@]34C)C1C[C@@H](O)[C@@H]2O ; C[C@]12CCC3C(C1CC[C@@H]2O)[C@@H](O)C[C@H]1C[C@@H](O)CC[C@]31C]

In some embodiments, the pharmaceutical composition includes an effective amount of a compound having the structure:

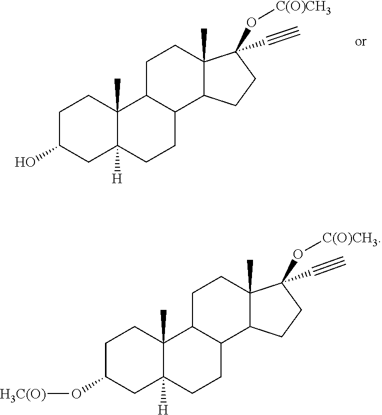

In some embodiments, the pharmaceutical composition includes an effective amount of a compound having the structure:

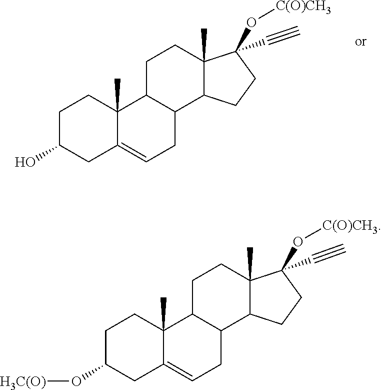

In some embodiments, the pharmaceutical composition includes 17-ethynyl-10R, 13S-dimethyl 2, 3, 4, 7, 8R, 95, 10, 11, 12, 13, 14S, 15, 16, 17-hexadecahydro-1H-cyclopenta[a]phenanthrene-3R, 7R, 17S-triol, which is represented by Formula 1. The compound of Formula 1 may also be referred to as Compound 1 or 17α-ethynylandrost-5-ene-3β,7β,17β-triol and is represented by the structure below.

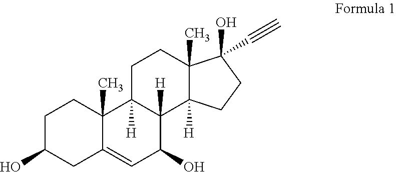

In some embodiments, the pharmaceutical composition includes (3S,5R,7S,8R,9S,10S,13S,14S,17R)-17-ethynyl-10,13-dimethylhexadecahydro-1H-cyclopenta[a]phenanthrene-3,7,17-triol, which is represented by Formula 2. The compound of Formula 2 may also be referred to as Compound 2, and is represented by the structure below.

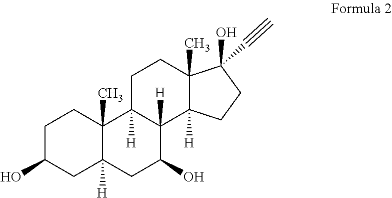

In some embodiments, a method is provided for to treat, reduce, prevent, or ameliorate a disease or condition in a subject. In some embodiments, the method to prevent a disease or condition in the subject may be by administration of a compound as described herein, or a pharmaceutical form thereof, and at least one pharmaceutically acceptable excipient. In some embodiments, the method to prevent a disease or condition in the subject may be by administration of Compound 1 or Compound 2, or a pharmaceutical form thereof, and at least one pharmaceutically acceptable excipient. In some embodiments, the method to prevent a disease or condition in the subject may be by administration of 17α-ethynylandrost-5-ene-3β,7β,17β-triol and at least one pharmaceutically acceptable excipient. In some embodiments, the method further includes measuring the biological age of the subject. In some embodiments, measuring the biological age of the subject is determining the chronological age of the subject and reversing or decreasing the rate of increase in the biological age of the subject, thereby preventing a disease or condition in the subject. In some embodiments, the disease or condition associated with a biological clock in a subject in need thereof is associated with DNA methylation levels in a subject.

In some embodiments, the disease or condition associated with biological clocks in the subject in need thereof is based on modulation of DNA methylation of genes associated with biological clocks. In some embodiments, the disease or condition associated with a biological clock in a subject in need thereof is associated with genes or genomic regions hypermethylated with age. In some embodiments, the disease or condition associated with a biological clock in a subject in need thereof is associated with genes or genomic regions hypomethylated with age. In some embodiments, the disease or condition associated with a biological clock in a subject in need thereof is associated with Tau phosphorylation. In some embodiments, the disease or condition associated with a biological clock in a subject in need thereof is associated with hyperglycemia. In some embodiments, the disease or condition associated with a biological clock in a subject in need thereof is associated with insulin resistance.

In some embodiments, the present disclosure provides a method of reducing DNA methylation in a subject in need thereof. In some embodiments, the method includes administering to the subject an effective amount of a compound as described herein, or a pharmaceutical form thereof, and at least one pharmaceutically acceptable excipient, thereby reducing DNA methylation in the subject. In some embodiments, the compound is Compound 1 or Compound 2, or a pharmaceutically acceptable salt thereof. In some embodiments, the compound is 17α-ethynylandrost-5-ene-3β,7β,17β-triol. The administering to the subject in need thereof has been shown to provide multiple beneficial responses to the subject. For example, in some embodiments, the administering reduces DNA methylation in the subject by at least 5% (e.g., at least 5, 6, 7, 8, 9, 10, 11, 12, 13, 14, or 15% or more or any value or range therein) as compared to a control measurement, e.g., as compared to DNA methylation in the subject prior to the administering (e.g., subject “baseline” DNA methylation). DNA methylation in the subject may be quantitatively and/or qualitatively evaluated by any standard technique in the art, e.g., as measured by a marker of relative global methylation as compared to a control, e.g., as measured by LINE-1 methylation as compared to a control. For example, in some embodiments, the administering reduces LINE-1 methylation in the subject by at least 5% (e.g., at least 5, 6, 7, 8, 9, 10, 11, 12, 13, 14, or 15% or more) as compared to a control measurement, e.g., as compared to LINE-1 methylation in the subject prior to the administering (e.g., e.g., subject baseline LINE-1 methylation). For example, in some embodiments, the administering may reduce LINE-1 methylation in the subject by at least 5%, at least 8%, at least 10% or at least 15% or more. In some embodiments, the administering may reduce LINE-1 methylation in the subject by about 5% to about 20%, about 6% to about 15%, or by about 8% to about 10%. In some embodiments, the improvement in symptoms related to DNA methylation may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of 17α-ethynylandrost-5-ene-3β,7β,17β-triol. For example, subjects may experience an improvement in symptoms or conditions related DNA methylation from approximately 5% to objectively normal after administration of 17α-ethynylandrost-5-ene-3β,7β,17β-triol and at least one pharmaceutically acceptable excipient.

In some embodiments, the subject may experience an improvement in symptoms or conditions related to genes or genomic regions hypermethylated with age after administration of a composition as described herein. In some embodiments, the subject may experience an improvement in symptoms or conditions related to genes or genomic regions hypermethylated with age after administration of 17α-ethynylandrost-5-ene-3β,7β,17β-triol. In some embodiments, the improvement in symptoms related to genes or genomic regions hypermethylated with age may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of 17α-ethynylandrost-5-ene-3β,7β,17β-triol. For example, subjects may experience an improvement in symptoms or conditions related to genes or genomic regions hypermethylated with age ranging from approximately 5% to 100% after administration of 17α-ethynylandrost-5-ene-3β,7β,17β-triol and at least one pharmaceutically acceptable excipient.

In some embodiments, the subject may experience an improvement in symptoms or conditions related to genes or genomic regions hypomethylated with age after administration of a composition as described herein. In some embodiments, the subject may experience an improvement in symptoms or conditions related to genes or genomic regions hypomethylated with age after administration of a composition as described herein. In some embodiments, the improvement in symptoms related to genes or genomic regions hypomethylated with age may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience an improvement in symptoms or conditions related to genes or genomic regions hypomethylated with age ranging from approximately 5% to 100% after administration of 17-ethynylandrost-5-ene-3β,7β,17β-triol and at least one pharmaceutically acceptable excipient.

In some embodiments, the subject may experience an improvement in symptoms or conditions related to hyperglycemia (which can lead to type I and type II diabetes) after administration of a composition as described herein. In some embodiments, the subject may experience an improvement in symptoms or conditions related to hyperglycemia (which can lead to type I and type II diabetes) after administration of 17α-ethynylandrost-5-ene-3β,7β,17β-triol. In some embodiments, the improvement in symptoms related to hyperglycemia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience an improvement in symptoms or conditions related to hyperglycemia ranging from approximately 5% to 100% after administration of 17α-ethynylandrost-5-ene-3β,7β,17β-triol and at least one pharmaceutically acceptable excipient.

In some embodiments, the subject may experience an improvement in symptoms or conditions related to hyperlipidemia (such as obesity-related conditions) after administration of a composition as described herein. In some embodiments, the subject may experience an improvement in symptoms or conditions related to hyperlipidemia (such as obesity-related conditions) after administration of 17α-ethynylandrost-5-ene-3β,7β,17β-triol. In some embodiments, the improvement in symptoms related to hyperlipidemia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of 17α-ethynylandrost-5-ene-3β,7β,17β-triol and at least one pharmaceutically acceptable excipient. For example, subjects may experience an improvement in symptoms or conditions related to hyperlipidemia ranging from approximately 5% to 100% after administration of 17α-ethynylandrost-5-ene-3β,7β,17β-triol and at least one pharmaceutically acceptable excipient.

In some embodiments, the subject may experience an improvement in symptoms or conditions related to insulin resistance after administration of a composition as described herein. In some embodiments, the subject may experience an improvement in symptoms or conditions related to insulin resistance after administration of 17α-ethynylandrost-5-ene-3β,7β,17β-triol. In some embodiments, the improvement in symptoms related to insulin resistance may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience an improvement in symptoms or conditions related to insulin resistance ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to an Alzheimer's Disease Composite Score after administration of a composition as described herein. In some embodiments, the subject may experience a reduction or decrease in symptoms related to an Alzheimer's Disease Composite Score after administration of 17α-ethynylandrost-5-ene-3β,7β,17β-triol. In some embodiments, the reduction or decrease in symptoms related to an Alzheimer's Disease Composite Score may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to Alzheimer's Disease Composite Score ranging from approximately 5% to 100% after administration of 17α-ethynylandrost-5-ene-3β,7β,17β-triol and at least one pharmaceutically acceptable excipient. In some embodiments, reducing or decreasing symptoms related to Alzheimer's Disease Composite Score may reduce other neuropsychological measures, including but not limited to, ADAS-Cog, MoCA, MMSE, CDR, QDRS, PDQ-9. In some embodiments, reducing or decreasing symptoms related to Alzheimer's Disease Composite Score may improve neuroimaging, including but not limited to, ASL, BOLD, MRS, task-based or resting fMRI, vMRI and FDG-PET.

In some embodiments, the subject may experience a reduction or decrease in symptoms related to CSF phosphorylated tau (“pTau”) after administration of a composition as described herein. In some embodiments, the subject may experience a reduction or decrease in symptoms related to pTau after administration of 17α-ethynylandrost-5-ene-3β,7β,17β-triol. In some embodiments, the reduction or decrease in symptoms related to pTau may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to pTau ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to leptin deficiency after administration of a composition as described herein. In some embodiments, the subject may experience a reduction or decrease in symptoms related to leptin deficiency after administration of 17α-ethynylandrost-5-ene-3β,7β,17β-triol. In some embodiments, the reduction or decrease in symptoms related to leptin deficiency may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to leptin deficiency ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to leptin resistance after administration of a composition as described herein. In some embodiments, the subject may experience a reduction or decrease in symptoms related to leptin resistance after administration of 17α-ethynylandrost-5-ene-3β,7β,17β-triol. In some embodiments, the reduction or decrease in symptoms related to leptin resistance may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to leptin resistance ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to DNA methylation after administration of a composition as described herein. In some embodiments, the subject may experience a reduction or decrease in symptoms related to DNA methylation after administration of 17α-ethynylandrost-5-ene-3β,7β,17β-triol. In some embodiments, the reduction or decrease in symptoms related to DNA methylation may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of 17α-ethynylandrost-5-ene-3β,7β,17β-triol and at least one pharmaceutically acceptable excipient. For example, subjects may experience reduction in symptoms related to DNA methylation ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to cardiovascular risk after administration of a composition as described herein. In some embodiments, the subject may experience a reduction or decrease in symptoms related to cardiovascular risk after administration of 17α-ethynylandrost-5-ene-3β,7β,17β-triol. In some embodiments, the reduction or decrease in symptoms related to cardiovascular risk may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to cardiovascular risk ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to a subject's epigenetic age (“DNA methylation phenoage”) after administration of a composition as described herein. In some embodiments, the subject may experience a reduction or decrease in symptoms related to DNA methylation phenoage after administration of 17α-ethynylandrost-5-ene-3β,7β,17β-triol. In some embodiments, the reduction or decrease in symptoms related to DNA methylation phenoage may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to DNA methylation phenoage ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to the subject's epigenetic clock (“DNA methylation skin blood clock”) after administration of a composition as described herein. In some embodiments, the subject may experience a reduction or decrease in symptoms related to the subject's DNA methylation skin blood clock after administration of 17α-ethynylandrost-5-ene-3β,7β,17β-triol. In some embodiments, the reduction or decrease in symptoms related to DNA methylation skin blood clock may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to DNA methylation skin blood clock ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to the subject's DunedinPACE clock after administration of a composition as described herein. In some embodiments, the subject may experience a reduction or decrease in symptoms related to the subject's DunedinPACE clock after administration of 17α-ethynylandrost-5-ene-3β,7β,17β-triol. In some embodiments, the reduction or decrease in symptoms related to DunedinPACE clock may be reduced by an amount equal to or greater than 75%, 80%, 85%, 90%, 99%, 100%, or ranges including and/or spanning the aforementioned values after administration of a composition as described herein. For example, subjects may experience reduction in symptoms related to DunedinPACE clock ranging from approximately 5% to 100% after administration of 17α-ethynylandrost-5-ene-3β,7β,17β-triol and at least one pharmaceutically acceptable excipient.

In some embodiments, the subject may experience a prevention, reduction or decrease in conditions or symptoms related to a cancer or tumor after administration of a composition as described herein. In some embodiments, the subject may experience a prevention, reduction or decrease in conditions or symptoms related to a cancer or tumor after administration of 17α-ethynylandrost-5-ene-3β,7β,17β-triol. In some embodiments, the prevention, reduction or decrease in conditions or symptoms related to a cancer or tumor may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience prevention, reduction or decrease in conditions or symptoms related to a cancer or tumor ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to atherosclerosis after administration of a composition as described herein. In some embodiments, the subject may experience a reduction or decrease in symptoms related to atherosclerosis after administration of 17α-ethynylandrost-5-ene-3β,7β,17β-triol. In some embodiments, the reduction or decrease in symptoms related to atherosclerosis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to atherosclerosis ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to schizophrenia after administration of a composition as described herein. In some embodiments, the subject may experience a reduction or decrease in symptoms related to schizophrenia after administration of 17α-ethynylandrost-5-ene-3β,7β,17β-triol. In some embodiments, the reduction or decrease in symptoms related to schizophrenia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to schizophrenia ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to an autoimmune disease after administration of a composition as described herein. In some embodiments, the subject may experience a reduction or decrease in symptoms related to an autoimmune disease after administration of 17α-ethynylandrost-5-ene-3β,7β,17β-triol. In some embodiments, the reduction or decrease in symptoms related to an autoimmune disease may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to an autoimmune disease ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to rheumatoid arthritis after administration of a composition as described herein. In some embodiments, the subject may experience a reduction or decrease in symptoms related to rheumatoid arthritis after administration of 17α-ethynylandrost-5-ene-3β,7β,17β-triol. In some embodiments, the reduction or decrease in symptoms related to rheumatoid arthritis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to rheumatoid arthritis ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to systemic lupus erythematosus after administration of a composition as described herein. In some embodiments, the subject may experience a reduction or decrease in symptoms related to systemic lupus erythematosus after administration of 17α-ethynylandrost-5-ene-3β,7β,17β-triol. In some embodiments, the reduction or decrease in symptoms related to systemic lupus erythematosus may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to systemic lupus erythematosus ranging from approximately 5% to 100% after administration of 17α-ethynylandrost-5-ene-3β,7β,17β-triol and at least one pharmaceutically acceptable excipient.

In some embodiments, the subject may experience a reduction or decrease in symptoms related to multiple sclerosis after administration of a composition as described herein. In some embodiments, the subject may experience a reduction or decrease in symptoms related to multiple sclerosis after administration of 17α-ethynylandrost-5-ene-3β,7β,17β-triol. In some embodiments, the reduction or decrease in symptoms related to multiple sclerosis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to multiple sclerosis ranging from approximately 5% to 100% after administration of 17α-ethynylandrost-5-ene-3β,7β,17β-triol and at least one pharmaceutically acceptable excipient.

In some embodiments, the subject may experience prevention or a reduction or decrease in the risk to ovum or sperm related to increased risks of offspring with autism spectrum disorder after administration of a composition as described herein. In some embodiments, the subject may experience prevention or a reduction or decrease in risk to ovum or sperm related to increased risks of offspring with symptoms related to autism spectrum disorder after administration of 17α-ethynylandrost-5-ene-3β,7β,17β-triol. In some embodiments, the reduction or decrease in ovum or sperm with increased risks of an offspring with symptoms related to autism spectrum disorder may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience prevention, reduction or decrease in ovum or sperm with increased risks of an offspring with symptoms related to autism spectrum disorder ranging from approximately 5% to 100% after administration of 17α-ethynylandrost-5-ene-3β,7β,17β-triol and at least one pharmaceutically acceptable excipient.

In some embodiments, the subject may experience prevention or a reduction or decrease in ovum or sperm with increased risks of an offspring with Down Syndrome after administration of a composition as described herein. In some embodiments, the subject may experience prevention or a reduction or decrease in ovum or sperm with increased risks of an offspring with Down Syndrome after administration of 17α-ethynylandrost-5-ene-3β,7β,17β-triol. In some embodiments, the reduction or decrease in symptoms related to Down Syndrome may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience prevention or reduction in ovum or sperm with increased risks of an offspring with Down Syndrome ranging from approximately 5% to 100% after administration of 17α-ethynylandrost-5-ene-3β,7β,17β-triol and at least one pharmaceutically acceptable excipient.

In several embodiments, the pharmaceutical composition includes a solid state form a compound as described herein. In several embodiments, the pharmaceutical compositions include a solid state form of 17α-ethynylandrost-5-ene-3β,7β,17β-triol. In several embodiments, the solid state form is crystalline 17α-ethynylandrost-5-ene-3β,7β,17β-triol. In several embodiments, the solid state form is crystalline 17α-ethynylandrost-5-ene-3β,7β,17β-triol substantially free of 17-ethynylandrost-5-ene-3β,7β,17β-triol in amorphous form.

In several embodiments, the solid state form is crystalline solvate 17α-ethynylandrost-5-ene-3β,7β,17β-triol. In several embodiments, the crystalline solvate is crystalline methanolate 17α-ethynylandrost-5-ene-3β,7β,17β-triol. In several embodiments, the crystalline solvate is crystalline ethanolate 17α-ethynylandrost-5-ene-3β,7β,17β-triol. In several embodiments, the crystalline solvate is crystalline hydrate 17α-ethynylandrost-5-ene-3β,7β,17β-triol.

In several embodiments, the crystalline solvate is Form III 17α-ethynylandrost-5-ene-3β,7β,17β-triol. In several embodiments, the crystalline solvate is Form IV 17α-ethynylandrost-5-ene-3β,7β,17β-triol. In several embodiments, the crystalline solvate is Form V 17α-ethynylandrost-5-ene-3β,7β,17β-triol.

In several embodiments, the solid-state form of 17α-ethynylandrost-5-ene-3β,7β,17β-triol is amorphous 17α-ethynylandrost-5-ene-3β,7β,17β-triol. In several embodiments, the amorphous 17α-ethynylandrost-5-ene-3β,7β,17β-triol substantially free of 17α-ethynylandrost-5-ene-3β,7β,17β-triol in solid state form.

In some embodiments, the compound or pharmaceutical composition described herein is administered orally. In some embodiments, the compound or pharmaceutical composition described herein is administered intravenously. In some embodiments, the compound or pharmaceutical composition described herein is administered topically. In some embodiments, 17α-ethynylandrost-5-ene-3β,7β,17β-triol is administered orally. In other embodiments, 17α-ethynylandrost-5-ene-3β,7β,17β-triol is administered intravenously. In other embodiments, 17α-ethynylandrost-5-ene-3β,7β,17β-triol is administered topically.

In some embodiments, a compound as described herein is administered as a formulation or a composition with at least one pharmaceutically acceptable excipient. In some embodiments, a compound as described herein is administered as a formulation or a composition with at least one pharmaceutically acceptable excipient and at least one pharmaceutically acceptable carrier. In some embodiments, 17α-ethynylandrost-5-ene-3β,7β,17β-triol is administered as a formulation with at least one pharmaceutically acceptable excipient. In some embodiments, 17α-ethynylandrost-5-ene-3β,7β,17β-triol is administered as a formulation with at least one pharmaceutically acceptable excipient and at least one pharmaceutically acceptable carrier. In some embodiments, 17α-ethynylandrost-5-ene-3β,7β,17β-triol is administered as a formulation with at least one pharmaceutically acceptable carrier. Other pharmaceutically acceptable excipients suitable for use in the compositions include absorption enhancing agents, acidifying agents, agents for modified release, alkalizing agents, antioxidants, buffering agents, chelating agents, coloring agents, complexing agents, emulsifying agents, flavoring agents, humectants, humidity-adjusting agents, pH-adjusting agents, preservatives, solubilizing agents, stabilizers, surface-active agents, suspending agents, sweetening agents, taste-masking agents, and wetting agents.

Formulations include compositions comprising 1, 2, 3, 4 or more pharmaceutically acceptable excipients or carriers. The compositions are used to prepare formulations suitable for human or animal use. Suitable administration routes for formulations include oral, rectal, nasal, transmucosal, topical (including buccal and sublingual), vaginal, rectal and parenteral (including subcutaneous, intramuscular, intravenous, intradermal, intrathecal, intraocular and epidural). In general, aqueous and non-aqueous liquid or cream formulations are delivered by a parenteral, oral or topical route. In other embodiments, such as the disclosure intermittent dosing methods, 17α-ethynylandrost-5-ene-3β,7β,17β-triol may be present as an aqueous or a non-aqueous liquid formulation or a solid formulation suitable for administration by any of the routes disclosed herein, e.g., oral, topical, buccal, sublingual, parenteral, inhaled aerosol or a depot such as a subcutaneous depot or an intraperitoneal or intramuscular depot. It will be appreciated that the preferred route may vary with, for example, the subject's pathological condition or weight or the subject's response to therapy with 17α-ethynylandrost-5-ene-3β,7β,17β-triol or other therapy that is used or that is appropriate to the circumstances.

The formulations include those suitable for the foregoing administration routes. The formulations may conveniently be presented in unit dosage form and may be prepared by any of the methods known in the art of pharmacy. Techniques, excipients and formulations generally are found in, e.g., Remington's Pharmaceutical Sciences, Mack Publishing Co., Easton, Pa. 2022, 23^{rd} edition, Adeboye et al., PDA J. Pharm. Sci. Tech. 1997 51:166-171, G. Cole, et al., editors, Pharmaceutical Coating Technology, 1995, Taylor & Francis, ISBN 0 136628915, H. A. Lieberman, et al., editors, Pharmaceutical Dosage Forms, 1992 2^{nd} revised edition, volumes 1 and 2, Marcel Dekker, ISBN 0824793870, J. T. Carstensen. Pharmaceutical Preformulation, 1998, pages 1-306, Technomic Publishing Co. ISBN 1566766907. Exemplary excipients for formulations include emulsifying wax, propyl gallate, citric acid, lactic acid, polysorbate 80, sodium chloride, isopropyl palmitate, glycerin, white petrolatum and other excipients disclosed herein.

Formulations, or compositions disclosed herein for use to make formulations suitable for administration by the routes disclosed herein optionally comprise an average particle size in the range of about 0.01 to about 500 microns, about 0.1 to about 100 microns or about 0.5 to about 75 microns. Average particle sizes include a range between 0.01 and 500 microns in 0.05 micron or in 0.1 micron or other increments, e.g., an average particle size of about 0.05, 0.1, 0.5, 1, 1.5, 2.0, 2.5, 3.0, 3.5, 4.0, 4.5, 5.0, 5.5, 6, 7, 8, 9, 10, 15, 20, 25, 30, 35, 40, 50, 60, 75, 85, 100, 120, etc. microns). When 17α-ethynylandrost-5-ene-3β,7β,17β-triol or compositions that comprise 17α-ethynylandrost-5-ene-3β,7β,17β-triol are used as intermediates to make a formulation, they may comprise one, two, three or more of these average particle sizes, or size ranges. In preparing any of the compositions or formulations that are disclosed herein and that comprise 17α-ethynylandrost-5-ene-3β,7β,17β-triol (and optionally one or more excipients and/or one or more carriers), one may optionally mill, sieve or otherwise granulate the compound or composition to obtain a desired particle size.

Non-limiting examples of fillers suitable for use in the compositions include lactose, microcrystalline cellulose, hydroxypropylcellulose, hydroxypropyl methylcellulose, methyl cellulose polymers hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, and collagen.

Non-limiting examples of diluents suitable for use in the compositions include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, and sugar.

Non-limiting examples of disintegrants suitable for use in the compositions include alginic acid or alginates, microcrystalline cellulose, low-substituted hydroxypropyl cellulose and other cellulose derivatives, croscarmellose sodium, crospovidone, polacrillin potassium, sodium starch glycolate, starch, pregelatinized starch, and carboxymethyl starch.

Non-limiting examples of binders suitable for use in the compositions include acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, polyethylene oxides, povidone, and pregelatinized starch.

Non-limiting examples of glidants and/or lubricants suitable for use in the compositions include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, and sodium acetate.

Non-limiting examples of antioxidants suitable for use in the compositions include ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, and derivatives of tocopherol.

In several embodiments, the pharmaceutically acceptable excipient is selected from sodium dodecyl sulfate, microcrystalline cellulose, magnesium stearate, and any combination of the foregoing. In several embodiments, the pharmaceutically acceptable excipient is sodium dodecyl sulfate.

In several embodiments, the pharmaceutical compositions are formulated into oral dosage forms. In several embodiments, the dosage forms can include capsules and tablets. In some embodiments, the dosage forms can include one or more different types of delayed release layers selected from sealant and/or enteric layers. For example, delayed release layers having different release rate characteristics can provide the dosage form with different overall drug release characteristics. In some such embodiments, the pharmaceutically acceptable excipient is a surface active agent. In several embodiments, the surface active agent is present in an amount sufficient to provide 90% dissolution of the pharmaceutical composition in water at ambient temperature after 30 min. In several embodiments, the surface active agent is sodium lauryl sulfate. In several embodiments, the pharmaceutical composition is a capsule or a tablet.

In several embodiments, the pharmaceutical compositions contain less than about 3% by weight of impurities.

In several embodiments, the pharmaceutical compositions contain less than about 5% by weight of 3β-hydroxy-androst-5-ene-7,17-dione.

In several embodiments, the pharmaceutical compositions include a pharmaceutically acceptable formulation of 17α-ethynylandrost-5-ene-3β,7β,17β-triol.

In several embodiments, the use is concurrent with a use of at least one additional medicament. In several embodiments, the additional medicament is administered at a delay time after a first administration of the composition. In several embodiments, the first administration may occur using a dosage schedule that is daily, weekly, monthly, or any combination of the foregoing. In several embodiments, the dosage schedule of the first administration may include one, two, three or more daily dosages of the composition. In several embodiments, the dosage schedule of the first administration may include one, two, three or more weekly dosages of the composition. In several embodiments, the dosage schedule of the first administration may include one, two, three or more monthly dosages of the composition. In several embodiments, the delay time is equal to or greater than about: 0.5, 1, 1.5, 2, 3, 4, 5, 6, 7, 8, 9, 10, 11, 12, 13, 14, 15, 16, 17, 18, 19, 20 years, or ranges including and/or spanning the aforementioned values. In several embodiments, the delay time is equal to or greater than 2 years. In some embodiments, the delay time is zero and the additional medicament is administered concurrently with the first administration of the composition. In several embodiments, the additional medicament is administered using a dosage schedule that is daily, weekly, monthly, or any combination of the foregoing. In several embodiments, the dosage schedule of the additional medicament may include one, two, three or more daily dosages of the composition. In several embodiments, the dosage schedule of the additional medicament may include one, two, three or more weekly dosages of the composition. In several embodiments, the dosage schedule of the additional medicament may include one, two, three or more monthly dosages of the composition.

Several embodiments of the present disclosure relate to the use of 17α-ethynylandrost-5-ene-3β,7β,17β-triol in the manufacture of a medicament for treating a neurodegenerative condition.

Aspects of the present disclosure relate to an in vitro screening method to identify a potential drug candidate. In some embodiments, the in vitro screening method may identify a potential drug candidate capable of treating, preventing, reducing, or ameliorating a disorder or disease. In some embodiments, the disorder or disease is a neurodegenerative disorder or disease. In some embodiments, the neurodegenerative disease or condition is dementia. As used herein, “drug candidate” refers to a specific molecule, compound, or therapeutic, whether of natural or synthetic origin, that has potential therapeutic effects and has been selected for further development and evaluation.

In some embodiments, the in vitro screening method may include providing a sample for stimulation. In some embodiments, the sample is a cell. In some embodiments, the sample is tissue. In some embodiments, the sample is blood. In some embodiments, the sample includes monocytes. In some embodiments, the sample includes microglia. In some embodiments, the monocytes include, but are not limited to, CX3CR1^{low}, CCR2^{pos}, Ly6C^{high}, PD-L1^{neg}, CD14⁺⁺, CD16⁺, CD14^{dim}, CD16⁺, CD16-CX3CR1^{high}, CCR2^{neg}, Ly6C^{low}, PD-L1^{pos}. In some embodiments, the cells are T cells or granulocytes. In some embodiments, the cells are NK cells or granulocytes. In some embodiments, the cell for stimulation may be selected from the group consisting of, but not limited to, THP-1 human monocytes, RAW 264.7 macrophages, 3T3-L1 adipocytes, SH-SY 5Y neuroblastoma, PC-12 pheochromocytoma and Lund human mesencephalic (LUHMES) cells.

In some embodiments, the in vitro screening method may include stimulating the cell with an agonist to induce a phenotype or a phenotypic change. In some embodiments, the phenotype may correspond to a phenotype of a cell or tissue affected by a neurodegenerative disease or disorder. In some embodiments, the neurodegenerative disorder or disease may be selected from the group consisting of, but not limited to, Alzheimer's disease, Parkinson's disease, amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

In some embodiments, the in vitro screening method may include contacting the one or more cells or tissue exhibiting the phenotype with the potential drug candidate. In some embodiments, the in vitro screening method may include contacting the one or more cells exhibiting the phenotype with the potential drug candidate in parallel in a high-throughput screening method. In other embodiments, the in vitro screening method may include contacting the one or more cells exhibiting the phenotype with one or more potential drug candidates in parallel in a high throughput screening method. In still other embodiments, the in vitro screening method may include contacting the one or more cells exhibiting the phenotype with the potential drug candidate sequentially.

In some embodiments, the in vitro screening method may include determining a responsive change in the cell phenotype. In some embodiments, the responsive change may be a decrease, reduction, or loss in the cell phenotype. In some embodiments, the phenotype may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after contacting the cell exhibiting the phenotype with the potential drug candidate.

In some embodiments, the phenotype is associated with DNA methylation in the subject. In some embodiments, the DNA methylation is at a CpG site. In some embodiments, the CpG site is selected from, but not limited to, AC073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAM DIC, FABP5P1, MCM10, ANP32E, ZNF268, ESPN, DHFR, U6, MTUS1, ATP1B3, or a combination thereof. In some embodiments, DNA methylation may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after contacting the cell exhibiting the phenotype with the potential drug candidate. In some embodiments, the DNA methylation is decreased by more than 50%. In some embodiments, the DNA methylation is decreased by more than 55%. In some embodiments, the DNA methylation is decreased by more than 60%.

In some embodiments, the drug candidate or compound as described herein is provided to a subject to treat, prevent, reduce, or ameliorate a disease or condition associated with DNA methylation. In some embodiments, the disease or condition is associated with TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab. In some embodiments, treating, preventing, reducing or ameliorating a disease or condition associated with DNA methylation may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values or establishing an objectively normal or disease-free condition after administration of the drug candidate or compound. For example, subjects may experience an improvement in symptoms or conditions related to TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/A bregions hypermethylated with age ranging from approximately 5% to 100% or to an objectively normal condition after administration of the drug candidate or compound and at least one pharmaceutically acceptable excipient.

Aspects of the disclosure relate to a method of diagnosing a patient with a disease or a condition. In some embodiments, the method includes providing a patient with a potential drug candidate or compound as described herein capable of treating, preventing, reducing, or ameliorating a disorder or disease. In some embodiments, the method includes identifying DNA methylation changes in the patient. In some embodiments, the method includes diagnosing the patient with a disease or condition associated with a biomarker associated with DNA methylation.

In some embodiments, identifying DNA methylation changes in the patient identifies the CpGs decreased by more than 50%. In some embodiments, identifying DNA methylation changes is correlated with one or more clinical changes. In some embodiments, the DNA methylation changes at a CpG site is selected from the group consisting of AC073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof.

In some embodiments, the disease or condition associated with a biomarker associated with DNA methylation is selected from the group consisting of TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab, or a combination thereof. In some embodiments, the disease or condition associated with a biomarker associated with DNA methylation is selected from the group consisting of ADAS-Cog11, ADCOMS, CDR, CSF glucose, CSF pTau/Ab, frontal lobe volume, subcortical grey matter thickness, GRC, heart rate, MoCA, PDQ-9, precuneus glutathione, QDRS, QDRS-behavior, QDRS-cognition, Systolic BP, Tau, TNFα, and weight. In some embodiments, the biomarker associated with DNA methylation is decreased in TNF, CDR, QRDS-cognition, wherein the subject QDRS-cognition is improved. In some embodiments, the DNA methylation change is a decrease of >50%. In some embodiments, the DNA methylation change is a decrease of >55%. In some embodiments, the DNA methylation change is a decrease of >60%.

In some embodiments, the subject may experience prevention or a reduction or decrease in obesity after administration of a drug candidate or compound as described herein. In some embodiments, the subject may experience prevention or a reduction in obesity after administration of the drug candidate or compound. In some embodiments, the reduction or decrease in symptoms related to obesity may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience prevention or reduction in obesity ranging from approximately 5% to 100% after administration of the drug candidate or compound and at least one pharmaceutically acceptable excipient.

In some embodiments, the subject may experience prevention or a reduction or decrease in ADCOMS after administration of a drug candidate or compound as described herein. In some embodiments, the subject may experience improvements in ADCOMS after administration of the drug candidate or compound. In some embodiments, the improvements related to ADCOMS may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values or objectively normal after administration of the drug candidate or compound as described herein. For example, subjects may experience improvements in ADCOMS ranging from approximately 5% to 100% or objectively normal after administration of the drug candidate or compound and at least one pharmaceutically acceptable excipient.

In some embodiments, the subject may experience prevention or a reduction or decrease in CDR after administration of a drug candidate or compound as described herein. In some embodiments, the subject may experience improvements in CDR after administration of the drug candidate or compound. In some embodiments, the improvements related to CDR may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in CDR ranging from approximately 5% to 100% after administration of the drug candidate or compound and at least one pharmaceutically acceptable excipient.

In some embodiments, the subject may experience prevention or a reduction or decrease in CSF glucose after administration of a drug candidate or compound as described herein. In some embodiments, the subject may experience improvements in CSF glucose after administration of the drug candidate or compound. In some embodiments, the improvements related to CSF glucose may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in CSF glucose ranging from approximately 5% to 100% or objectively normal after administration of the drug candidate or compound and at least one pharmaceutically acceptable excipient.

In some embodiments, the subject may experience prevention or a reduction or decrease in CSF pTau/Aβ after administration of a drug candidate or compound as described herein. In some embodiments, the subject may experience improvements in CSF pTau/Aβ after administration of the drug candidate or compound. In some embodiments, the improvements related to CSF pTau/Aβ may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in CSF pTau/Aβ ranging from approximately 5% to 100% or objectively normal after administration of the drug candidate or compound and at least one pharmaceutically acceptable excipient.

In some embodiments, the subject may experience prevention or a reduction or decrease in frontal lobe volume after administration of a drug candidate or compound as described herein. In some embodiments, the subject may experience improvements in frontal lobe volume after administration of the drug candidate or compound. In some embodiments, the improvements related to frontal lobe volume may be improved by an amount equal to or greater than 75%, 80%, 85%, 90%, 99%, 100%, or ranges including and/or spanning the aforementioned values after administration of the drug candidate or compound as described herein. For example, subjects may experience improvements in frontal lobe volume ranging from approximately 5% to 100% or objectively normal after administration of the drug candidate or compound and at least one pharmaceutically acceptable excipient.

In some embodiments, the subject may experience prevention or a reduction or decrease in subcortical grey matter thickness after administration of a drug candidate or compound as described herein. In some embodiments, the subject may experience improvements in subcortical grey matter thickness after administration of the drug candidate or compound. In some embodiments, the improvements related to subcortical grey matter thickness may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in subcortical grey matter thickness ranging from approximately 5% to 100% or objectively normal after administration of the drug candidate or compound and at least one pharmaceutically acceptable excipient.

In some embodiments, the subject may experience prevention or a reduction or decrease in GRC after administration of a drug candidate or compound as described herein. In some embodiments, the subject may experience improvements in GRC after administration of the drug candidate or compound. In some embodiments, the improvements related to GRC may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in GRC ranging from approximately 5% to 100% or objectively normal after administration of the drug candidate or compound and at least one pharmaceutically acceptable excipient.

In some embodiments, the subject may experience prevention or a reduction or decrease in heart rate after administration of a drug candidate or compound as described herein. In some embodiments, the subject may experience improvements in heart rate after administration of the drug candidate or compound. In some embodiments, the improvements related to heart rate may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in heart rate ranging from approximately 5% to 100% or objectively normal after administration of the drug candidate or compound and at least one pharmaceutically acceptable excipient.

In some embodiments, the subject may experience prevention or a reduction or decrease in M oCA after administration of a drug candidate or compound as described herein. In some embodiments, the subject may experience improvements in MoCA after administration of the drug candidate or compound. In some embodiments, the improvements related to M oCA may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in M oCA ranging from approximately 5% to 100% or objectively normal after administration of the drug candidate or compound and at least one pharmaceutically acceptable excipient.

In some embodiments, the subject may experience prevention or a reduction or decrease in precuneus glutathione after administration of a drug candidate or compound as described herein. In some embodiments, the subject may experience improvements in precuneus glutathione after administration of the drug candidate or compound. In some embodiments, the improvements related to precuneus glutathione may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in precuneus glutathione ranging from approximately 5% to 100% or objectively normal after administration of the drug candidate or compound and at least one pharmaceutically acceptable excipient.

In some embodiments, the subject may experience prevention or a reduction or decrease in QDRS after administration of a drug candidate or compound as described herein. In some embodiments, the subject may experience improvements in QDRS after administration of the drug candidate or compound. In some embodiments, the improvements related to QDRS may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in QDRS ranging from approximately 5% to 100% or objectively normal after administration of the drug candidate or compound and at least one pharmaceutically acceptable excipient.

In some embodiments, the subject may experience prevention or a reduction or decrease in QDRS-behavior after administration of a drug candidate or compound as described herein. In some embodiments, the subject may experience improvements in QDRS-behavior after administration of the drug candidate or compound. In some embodiments, the improvements related to QDRS-behavior may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in QDRS-behavior ranging from approximately 5% to 100% or objectively normal after administration of the drug candidate or compound and at least one pharmaceutically acceptable excipient.

In some embodiments, the subject may experience prevention or a reduction or decrease in QDRS-behavior after administration of a drug candidate or compound as described herein. In some embodiments, the subject may experience improvements in QDRS-behavior after administration of the drug candidate or compound. In some embodiments, the improvements related to QDRS-behavior may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in QDRS-behavior ranging from approximately 5% to 100% or objectively normal after administration of the drug candidate or compound and at least one pharmaceutically acceptable excipient.

In some embodiments, the subject may experience prevention or a reduction or decrease in QDRS-cognition after administration of a drug candidate or compound as described herein. In some embodiments, the subject may experience improvements in QDRS-cognition after administration of the drug candidate or compound. In some embodiments, the improvements related to QDRS-cognition may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in QDRS-cognition ranging from approximately 5% to 100% or objectively normal after administration of the drug candidate or compound and at least one pharmaceutically acceptable excipient.

In some embodiments, the subject may experience prevention or a reduction or decrease in systolic blood pressure after administration of a drug candidate or compound as described herein. In some embodiments, the subject may experience improvements in systolic blood pressure after administration of the drug candidate or compound. In some embodiments, the improvements related to systolic blood pressure may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in systolic blood pressure ranging from approximately 5% to 100% or objectively normal after administration of the drug candidate or compound and at least one pharmaceutically acceptable excipient.

In some embodiments, the subject may experience prevention or a reduction or decrease in Tau after administration of a drug candidate or compound as described herein. In some embodiments, the subject may experience improvements in Tau after administration of the drug candidate or compound. In some embodiments, the improvements related to Tau may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in Tau ranging from approximately 5% to 100% or objectively normal after administration of the drug candidate or compound and at least one pharmaceutically acceptable excipient.

In some embodiments, the subject may experience prevention or a reduction or decrease in TNFα after administration of a drug candidate or compound as described herein. In some embodiments, the subject may experience improvements in TNFα after administration of the drug candidate or compound. In some embodiments, the improvements related to TNFα may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in TNFα ranging from approximately 5% to 100% or objectively normal after administration of the drug candidate or compound and at least one pharmaceutically acceptable excipient.

In some embodiments, the subject may experience prevention or a reduction or decrease in weight after administration of a drug candidate or compound as described herein. In some embodiments, the subject may experience improvements in weight after administration of the drug candidate or compound. In some embodiments, the improvements related to weight may be improved by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience improvements in weight ranging from approximately 5% to 100% or objectively normal after administration of the drug candidate or compound and at least one pharmaceutically acceptable excipient.

In some embodiments, the subject may experience a modification to their insulin levels after administration of a compound as described herein. In some embodiments, the subject may experience a modification that increases fasting insulin and HOMA2-% B in a subject. In some embodiments, the subject may experience an insulin modification that decreases HOMA2-% S without hypoglycemia. In some embodiments, the subject may experience an insulin modification that increases insulin and HOMA2-% B cell function and decreases HOMA2% insulin sensitivity. In some embodiments, the modification to insulin may be in an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience a modification in insulin levels ranging from approximately 5% to 100% or objectively normal after administration of the drug candidate or compound and at least one pharmaceutically acceptable excipient.

In some embodiments, a subject may experience one or more improvements to a neuropsychiatric condition after being administered to a compound as described herein. In some embodiments, the one or more neuropsychiatric conditions includes sleep and appetite. In some embodiments, an improvement in appetite is correlated with decreased Cog12, CDR SB, and ADCOMS. In some embodiments, an improvement in sleep is correlated with decreased CGIC and decreased TNFα. In some embodiments, the one or more improvements to a neuropsychiatric condition may be in an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate or compound as described herein. For example, subjects may experience one or more improvements to a neuropsychiatric condition ranging from approximately 5% to 100% or objectively normal after administration of the drug candidate or compound and at least one pharmaceutically acceptable excipient.

Accordingly, some aspects described relate to the following numbered alternatives:
1. A method to treat, reduce, or ameliorate a disease or condition associated with biological clocks in a subject in need thereof, the method comprising administering to the subject 17α-ethynylandrost-5-ene-3β,7β,17β-triol.
2. The method of alternative 1, wherein the disease or condition associated with biological clocks in the subject in need thereof is based on modulation of DNA methylation of genes associated with biological clocks.
3. The method of alternative 1 or 2, wherein the disease or condition associated with a biological clock in the subject in need thereof is associated with genes or genomic regions hypermethylated with age.
4. The method of any one of alternatives 1 to 3, wherein the disease or condition associated with a biological clock in the subject in need thereof is associated with genes or genomic regions hypomethylated with age.
5. The method of any one of alternatives 1 to 4, wherein the disease or condition associated with a biological clock in a subject in need thereof is associated with Tau phosphorylation.
6. The method of any one of alternatives 1 to 5, wherein the disease or condition associated with a biological clock in a subject in need thereof is associated with hyperglycemia.
7. The method of any one of alternatives 1 to 6, wherein the disease or condition associated with a biological clock in a subject in need thereof is associated with insulin resistance.
8. The method of any one of alternatives 1 to 7, wherein the disease or condition associate with biological clock is mild cognitive impairment or late onset Alzheimer's disease.
9. The method of any one of alternatives 1 to 8, wherein administering to the subject 17α-ethynylandrost-5-ene-3β,7β,17β-triol and at least one pharmaceutically acceptable excipient decreases a subject's Alzheimer's Disease Composite Score.
10. The method of alternative 9, wherein the subject experiences a decrease between about a 5% to about a 100% reduction in symptoms connected to Alzheimer's Disease Composite Score or objectively normal after administration of 17α-ethynylandrost-5-ene-3β,7β,17β-triol and at least one pharmaceutically acceptable excipient.
11. The method of any one of alternatives 1 to 8, wherein administering to the subject 17α-ethynylandrost-5-ene-3β,7β,17β-triol and at least one pharmaceutically acceptable excipient decreases pTau in the subject.
12. The method of alternative 11, wherein the subject experiences a decrease between about a 5% to about a 100% reduction in conditions or symptoms connected to pTau or objectively normal after administration of 17α-ethynylandrost-5-ene-3β,7β,17β-triol and at least one pharmaceutically acceptable excipient.
13. The method of any one of alternatives 1 to 8, wherein administering to the subject 17α-ethynylandrost-5-ene-3β,7β,17β-triol and at least one pharmaceutically acceptable excipient decreases leptin in the subject.
14. The method of alternative 13, wherein the subject experiences an increase between about a 5% to about a 100% in conditions or symptoms connected to leptin or objectively normal after administration of 17α-ethynylandrost-5-ene-3β,7β,17β-triol and at least one pharmaceutically acceptable excipient.
15. The method of any one of alternatives 1 to 8, wherein administering to the subject 17α-ethynylandrost-5-ene-3β,7β,17β-triol and at least one pharmaceutically acceptable excipient decreases DNA methylation in the subject.
16. The method of alternative 15, wherein the subject experiences a decrease between about a 5% to about a 100% reduction in conditions or symptoms connected to DNA methylation or objectively normal after administration of 17α-ethynylandrost-5-ene-3β,7β,17β-triol and at least one pharmaceutically acceptable excipient.
17. The method of any one of alternatives 1 to 8, wherein administering to the subject 17α-ethynylandrost-5-ene-3β,7β,17β-triol and at least one pharmaceutically acceptable excipient decreases cardiovascular risk in the subject.
18. The method of alternative 17, wherein the subject experiences a decrease between about a 5% to about a 100% reduction in conditions or symptoms connected to cardiovascular risk or objectively normal after administration of 17α-ethynylandrost-5-ene-3β,7β,17β-triol and at least one pharmaceutically acceptable excipient.
19. The method of any one of alternatives 1 to 8, wherein administering to the subject 17α-ethynylandrost-5-ene-3β,7β,17β-triol and at least one pharmaceutically acceptable excipient decreases DNA methylation age in the subject.
20. The method of alternative 19, wherein the subject experiences a decrease between about a 5% to about a 100% reduction in conditions or symptoms connected to DNA methylation or objectively normal after administration of 17α-ethynylandrost-5-ene-3β,7β,17β-triol and at least one pharmaceutically acceptable excipient.
21. The method of any one of alternatives 1 to 8, wherein administering to the subject 17α-ethynylandrost-5-ene-3β,7β,17β-triol and at least one pharmaceutically acceptable excipient decreases DNA methylation phenoage in the subject.
22. The method of alternative 21, wherein the subject experiences a decrease between about a 5% to about a 100% reduction in conditions or symptoms connected to DNA methylation phenoage or objectively normal after administration of 17α-ethynylandrost-5-ene-3β,7β,17β-triol and at least one pharmaceutically acceptable excipient.
23. The method of any one of alternatives 1 to 8, wherein administering to the subject 17α-ethynylandrost-5-ene-3β,7β,17β-triol and at least one pharmaceutically acceptable excipient decreases DNA methylation skin blood clock in the subject.
24. The method of alternative 23, wherein the subject experiences a decrease between about a 5% to about a 100% reduction in conditions or symptoms connected to DNA methylation skin blood clock or objectively normal after administration of 17α-ethynylandrost-5-ene-3β,7β,17β-triol and at least one pharmaceutically acceptable excipient.
25. The method of any one of alternatives 1 to 8, wherein administering to the subject 17α-ethynylandrost-5-ene-3β,7β,17β-triol and at least one pharmaceutically acceptable excipient decreases a subject's CDR Score.
26. The method of alternative 25, wherein the subject experiences a decrease between about a 5% to about a 100% reduction in symptoms connected to CDR Score or objectively normal after administration of 17α-ethynylandrost-5-ene-3β,7β,17β-triol and at least one pharmaceutically acceptable excipient.
27. The method of any one of alternatives 1 to 8, wherein administering to the subject 17α-ethynylandrost-5-ene-3β,7β,17β-triol and at least one pharmaceutically acceptable excipient decreases a subject's ADAS-Cog Score.
28. The method of alternative 27, wherein the subject experiences a decrease between about a 5% to about a 100% reduction in symptoms connected to ADAS-Cog Score or objectively normal after administration of 17α-ethynylandrost-5-ene-3β,7β,17β-triol and at least one pharmaceutically acceptable excipient.
29. The method of any one of alternatives 1 to 8, wherein administering to the subject 17α-ethynylandrost-5-ene-3β,7β,17β-triol and at least one pharmaceutically acceptable excipient decreases a subject's QDRS Score.
30. The method of alternative 29, wherein the subject experiences a decrease between about a 5% to about a 100% reduction in symptoms connected to QDRS Score or objectively normal after administration of 17α-ethynylandrost-5-ene-3β,7β,17β-triol and at least one pharmaceutically acceptable excipient.
31. The method of any one of alternatives 1 to 8, wherein administering to the subject 17α-ethynylandrost-5-ene-3β,7β,17β-triol and at least one pharmaceutically acceptable excipient increases a subject's MMSE Score.
32. The method of alternative 31, wherein the subject experiences a decrease between about a 5% to about a 100% reduction in symptoms connected to MM SE Score or objectively normal after administration of 17α-ethynylandrost-5-ene-3β,7β,17β-triol and at least one pharmaceutically acceptable excipient.
33. The method of any one of alternatives 1 to 8, wherein administering to the subject 17α-ethynylandrost-5-ene-3β,7β,17β-triol and at least one pharmaceutically acceptable excipient decreases a subject's MoCA Score.
34. The method of alternative 33, wherein the subject experiences a decrease between about a 5% to about a 100% reduction in symptoms connected to MoCA Score or objectively normal after administration of 17α-ethynylandrost-5-ene-3β,7β,17β-triol and at least one pharmaceutically acceptable excipient.
35. The method of any one of alternatives 1 to 8, wherein administering to the subject 17α-ethynylandrost-5-ene-3β,7β,17β-triol and at least one pharmaceutically acceptable excipient decreases a subject's GRC Score.
36. The method of alternative 35, wherein the subject experiences a decrease between about a 5% to about a 100% reduction in symptoms connected to GRC Score or objectively normal after administration of 17-ethynylandrost-5-ene-3β,7β,17β-triol and at least one pharmaceutically acceptable excipient.
37. The method of any one of alternatives 1 to 8, wherein administering to the subject 17α-ethynylandrost-5-ene-3β,7β,17β-triol and at least one pharmaceutically acceptable excipient decreases a subject's CGIC Score.
38. The method of alternative 37, wherein the subject experiences a decrease between about a 5% to about a 100% reduction in symptoms connected to CGIC Score or objectively normal after administration of 17α-ethynylandrost-5-ene-3β,7β,17β-triol and at least one pharmaceutically acceptable excipient.
39. The method of any one of alternatives 1 to 8, wherein administering to the subject 17α-ethynylandrost-5-ene-3β,7β,17β-triol and at least one pharmaceutically acceptable excipient decreases a subject's ADL Score.
40. The method of alternative 39, wherein the subject experiences a decrease between about a 5% to about a 100% reduction in symptoms connected to ADL Score or objectively normal after administration of 17α-ethynylandrost-5-ene-3β,7β,17β-triol and at least one pharmaceutically acceptable excipient.
41. The method of any one of alternatives 1 to 8, wherein administering to the subject 17α-ethynylandrost-5-ene-3β,7β,17β-triol and at least one pharmaceutically acceptable excipient decreases a subject's PDQ9 Score.
42. The method of alternative 41, wherein the subject experiences a decrease between about a 5% to about a 100% reduction in symptoms connected to PDQ9 Score or objectively normal after administration of 17α-ethynylandrost-5-ene-3β,7β,17β-triol and at least one pharmaceutically acceptable excipient.
43. The method of any one of alternatives 1 to 42, wherein the 17α-ethynylandrost-5-ene-3β,7β,17β-triol is administered orally.
44. The method of any one of alternatives 1 to 42, wherein the 17α-ethynylandrost-5-ene-3β,7β,17β-triol is administered intravenously.
45. The method of any one of alternatives 1 to 44, wherein the subject has a waist to hip ratio greater than or equal to approximately 0.90.
46. The method of any one of alternatives 1 to 44, wherein the subject has a waist to hip ratio greater than or equal to approximately 0.95.
47. The method of any one of Alternatives 1 to 46, wherein the 17α-ethynylandrost-5-ene-3b,7b,17b-triol is a solid state form of 17α-ethynylandrost-5-ene-3b,7b,17b-triol.
48. The method of Alternative 47, wherein the solid state form of 17α-ethynylandrost-5-ene-3b,7b,17b-triol is crystalline solvate of 17α-ethynylandrost-5-ene-3b, 7b,17b-triol.
49. The method of Alternative 48, wherein the crystalline solvate is crystalline methanolate 17α-ethynylandrost-5-ene-3b,7b,17b-triol.
50. The method of Alternative 49, wherein the crystalline solvate is crystalline ethanolate 17α-ethynylandrost-5-ene-3b, 7b,17b-triol.
51. The method of Alternative 50, wherein the crystalline solvate is crystalline hydrate 17α-ethynylandrost-5-ene-3b, 7b,17b-triol.
52. The method of Alternative 50, wherein the crystalline solvate is Form III 17α-ethynylandrost-5-ene-3b,7b,17b-triol.
53. The method of Alternative 50, wherein the crystalline solvate is Form IV 17α-ethynylandrost-5-ene-3b, 7b,17b-triol.
54. The method of Alternative 50, wherein the crystalline solvate is Form V 17α-ethynylandrost-5-ene-3b,7b,17b-triol.
55. The method of Alternative 49, wherein the solid state form of 17α-ethynylandrost-5-ene-3b,7b,17b-triol is amorphous 17α-ethynylandrost-5-ene-3b,7b,17b-triol.
56. The method of any one of Alternatives 1 to 55, wherein the pharmaceutical composition contains less than about 3% by weight of impurities.
57. An in vitro screening method to identify a potential drug candidate or compound capable of treating, preventing, reducing, or ameliorating a disorder or disease, comprising: (i) providing a sample for stimulation selected from the group consisting of a cell, tissue, blood, monocytes, microglia, macrophages, adipocytes, neuroblastoma, pheochromocytoma, and Lund human mesencephalic (LUHMES) cells; (ii) stimulating the sample with an agonist to induce a phenotype or phenotypic reaction, wherein the phenotype or phenotypic reaction substantially corresponds to a disease or condition associated with at least one DNA methylation at a CpG site in a region of DNA; (iii) contacting the one or more cells exhibiting the phenotype or phenotypic reaction with one or more potential drug candidate or compounds; (iv) determining a responsive change in the phenotype of the sample; and (v) providing the drug candidate or compound to a subject in need thereof to treat, reduce, prevent, or ameliorate a disease or condition associated with the DNA methylation in the subject.
58. The method of alternative 57, wherein the at least one DNA methylation at the CpG site is selected from the group consisting of AC073869.20, SP100, KCNQ1DN, DBNDD2, CEP112, CEP85L, SPDYE4, ZNF211, NR3C1, HLA-L, TPP2, SLC26A1, SL C37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof.
59. The method of alternative 57 or 58, wherein the phenotype or phenotypic reaction is selected from the group consisting of TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab, or a combination thereof.
60. The method of alternative 59, wherein the phenotype or phenotypic reaction is selected from the group consisting of ADAS-Cog11, ADCOMS, CDR, CSF glucose, CSF pTau/Ab, frontal lobe volume, subcortical grey matter thickness, GRC, Heart rate, MoCA, PDQ-9, precuneus glutathione, QDRS, QDRS-behavior, QDRS-cognition, Systolic BP, Tau, TNFα, and weight.
61. The method of any one of alternatives 57 to 60, wherein the DNA methylation change is a decrease of >50%.
62. The method of any one of alternatives 57 to 60, wherein the DNA methylation change is a decrease of >55%.
63. The method of any one of alternatives 57 to 60, wherein the DNA methylation change is a decrease of >60%.
64. The method of alternative 59, wherein the phenotype or phenotypic reaction is decreased in TNF, CDR, QRDS-cognition, wherein the subject QDRS-cognition is improved.
65. The method of any one of alternatives 57 to 64, wherein the responsive change is a decrease or loss in the phenotype and the decrease or loss is indicative that the potential drug candidate or compound is capable of preventing, reducing, or ameliorating a neurodegenerative disorder or disease.
66. The method of alternative 65, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.
67. The method of alternative 65 or 66, wherein the neurodegenerative disorder or disease is Parkinson's disease.
68. The method of alternative 65 or 66, wherein the neurodegenerative disorder is Alzheimer's disease.
69. The method of any one of alternatives 57 to 68, wherein the CpGs sites are interconnected with genes associated with Alzheimer's disease and related dementias.
70. The method of alternative 69, wherein the disease or condition associated with inflammatory TNF signaling.
71. The method of any one of alternatives 57 to 69, wherein the disease or condition associated with DNA methylation associated with Tau phosphorylation.
72. The method of any one of alternatives 57 to 69, wherein the disease or condition associated with DNA methylation associated with hyperglycemia.
73. The method of any one of alternatives 57 to 69, wherein the disease or condition associated with DNA methylation associated with insulin resistance.
74. The method of any one of alternatives 57 to 69, wherein the disease or condition associated with DNA methylation associated with obesity.
75. The method of any one of 57 to 69, wherein the disease or condition associated with DNA methylation is associated with a subject's ADAS-Cog11.
76. The method of any one of 57 to 69, wherein the disease or condition associated with DNA methylation is associated with a subject's ADCOMS.
77. The method of any one of 57 to 69, wherein the disease or condition associated with DNA methylation is associated with a subject's CDR.
78. The method of any one of 57 to 69, wherein the disease or condition associated with DNA methylation is associated with a subject's CSF glucose.
79. The method of any one of 57 to 69, wherein the disease or condition associated with DNA methylation is associated with a subject's CSF pTau/Aβ.
80. The method of any one of 57 to 69, wherein the disease or condition associated with DNA methylation is associated with a subject's frontal lobe volume.
81. The method of any one of 57 to 69, wherein the disease or condition associated with DNA methylation is associated with a subject's subcortical grey matter thickness.
82. The method of any one of 57 to 69, wherein the disease or condition associated with DNA methylation is associated with a subject's GRC.
83. The method of any one of 57 to 69, wherein the disease or condition associated with DNA methylation is associated with a subject's heart rate.
84. The method of any one of 57 to 69, wherein the disease or condition associated with DNA methylation is associated with a subject's MoCA.
85. The method of any one of 57 to 69, wherein the disease or condition associated with DNA methylation is associated with a subject's MoCA.
86. The method of any one of 57 to 69, wherein the disease or condition associated with DNA methylation is associated with a subject's PDQ-9.
87. The method of any one of 57 to 69, wherein the disease or condition associated with DNA methylation is associated with a subject's precuneus glutathione.
88. The method of any one of 57 to 69, wherein the disease or condition associated with DNA methylation is associated with a subject's QDRS.
89. The method of any one of 57 to 69, wherein the disease or condition associated with DNA methylation is associated with a subject's QDRS-behavior.
90. The method of any one of 57 to 69, wherein the disease or condition associated with DNA methylation is associated with a subject's QDRS-cognition.
91. The method of any one of 57 to 69, wherein the disease or condition associated with DNA methylation is associated with a subject's systolic blood pressure.
92. The method of any one of 57 to 69, wherein the disease or condition associated with DNA methylation is associated with a subject's Tau.
93. The method of any one of 57 to 69, wherein the disease or condition associated with DNA methylation is associated with a subject's TNFα.
94. The method of any one of 57 to 69, wherein the disease or condition associated with DNA methylation is associated with a subject's weight.
95. A method of diagnose a patient with a disease or a condition, the method comprising: (i) providing a patient with a potential drug candidate or compound capable of treating, preventing, reducing, or ameliorating a disorder or disease; (ii) identifying DNA methylation changes in the patient; and (iii) diagnosing the patient with a disease or condition associated with a biomarker associated with DNA methylation.
96. The method of alternative 95, wherein identifying DNA methylation changes in the patient identifies a CpGs decreased by more than 50%.
97. The method of alternative 95 or 96, wherein identifying DNA methylation changes is correlated with one or more clinical changes.
98. The method of any one of alternatives 95 or 97, wherein the DNA methylation changes at a CpG site is selected from the group consisting of A C073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof.
99. The method of any one of alternatives 95 or 98, wherein the disease or condition associated with a biomarker associated with DNA methylation is selected from the group consisting of TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab, or a combination thereof.
100. The method of alternative 99, wherein the disease or condition associated with a biomarker associated with DNA methylation is selected from the group consisting of ADAS-Cog11, ADCOMS, CDR, CSF glucose, CSF pTau/Ab, frontal lobe volume, subcortical grey matter thickness, GRC, Heart rate, MoCA, PDQ-9, precuneus glutathione, QDRS, QDRS-behavior, QDRS-cognition, Systolic BP, Tau, TNFα, and weight.
101. The method of alternative 99, wherein the biomarker associated with DNA methylation is decreased in TNF, CDR, QRDS-cognition, wherein the subject QDRS-cognition is improved.
102. The method of any one of alternatives 95 to 101, wherein the DNA methylation change is a decrease of >50%.
103. The method of any one of alternatives 95 to 101, wherein the DNA methylation change is a decrease of >55%.
104. The method of any one of alternatives 95 to 101, wherein the DNA methylation change is a decrease of >60%.
105. The method of any one of alternatives 95 to 104, wherein the disease or condition is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.
106. The method of alternative 105, wherein the neurodegenerative disorder or disease is Parkinson's disease.
107. The method of alternative 105, wherein the neurodegenerative disorder is Alzheimer's disease.
108. The method of any one of alternatives 95 to 107, wherein the CpGs sites are interconnected with genes associated with Alzheimer's disease and related dementias.
109. The method of any one of alternatives 95 to 108, wherein the disease or condition is associated with inflammatory TNF signaling.
110. The method of any one of alternatives 95 to 108, wherein the disease or condition associated with DNA methylation is associated with Tau phosphorylation.
111. The method of any one of alternatives 95 to 108, wherein the disease or condition associated with DNA methylation is associated with hyperglycemia.
112. The method of any one of alternatives 95 to 108, wherein the disease or condition associated with DNA methylation associated with insulin resistance.
113. The method of any one of alternatives 95 to 108, wherein the disease or condition associated with DNA methylation is associated with obesity.
114. The method of any one of alternatives 95 to 108, wherein the disease or condition associated with DNA methylation is associated with a subject's ADAS-Cog11.
115. The method of any one of alternatives 95 to 108, wherein the disease or condition associated with DNA methylation is associated with a subject's ADCOMS.
116. The method of any one of alternatives 95 to 108, wherein the disease or condition associated with DNA methylation is associated with a subject's CDR.
117. The method of any one of alternatives 95 to 108, wherein the disease or condition associated with DNA methylation is associated with a subject's CSF glucose.
118. The method of any one of alternatives 95 to 108, wherein the disease or condition associated with DNA methylation is associated with a subject's CSF pTau/Aβ.
119. The method of any one of alternatives 95 to 108, wherein the disease or condition associated with DNA methylation is associated with a subject's frontal lobe volume.
120. The method of any one of alternatives 95 to 108, wherein the disease or condition associated with DNA methylation is associated with a subject's subcortical grey matter thickness.
121. The method of any one of alternatives 95 to 108, wherein the disease or condition associated with DNA methylation is associated with a subject's GRC.
122. The method of any one of alternatives 95 to 108, wherein the disease or condition associated with DNA methylation associated with a subject's heart rate.
123. The method of any one of alternatives 95 to 108, wherein the disease or condition associated with DNA methylation is associated with a subject's MoCA.
124. The method of any one of alternatives 95 to 108, wherein the disease or condition associated with DNA methylation is associated with a subject's MoCA.
125. The method of any one of alternatives 95 to 108, wherein the disease or condition associated with DNA methylation is associated with a subject's PDQ-9.
126. The method of any one of alternatives 95 to 108, wherein the disease or condition associated with DNA methylation is associated with a subject's precuneus glutathione.
127. The method of any one of alternatives 95 to 108, wherein the disease or condition associated with DNA methylation is associated with a subject's QDRS.
128. The method of any one of alternatives 95 to 108, wherein the disease or condition associated with DNA methylation is associated with a subject's QDRS-behavior.
129. The method of any one of alternatives 95 to 108, wherein the disease or condition associated with DNA methylation is associated with a subject's QDRS-cognition.
130. The method of any one of alternatives 95 to 108, wherein the disease or condition associated with DNA methylation is associated with a subject's systolic blood pressure.
131. The method of any one of alternatives 95 to 108, wherein the disease or condition associated with DNA methylation is associated with a subject's Tau.
132. The method of any one of alternatives 95 to 108, wherein the disease or condition associated with DNA methylation is associated with a subject's TNFα.
132. The method of any one of alternatives 95 to 108, wherein the disease or condition associated with DNA methylation is associated with a subject's weight.
133. The method of any one of alternatives 95 to 108, wherein administering to the subject the drug candidate or compound decreases a subject's CDR Score.
134. The method of alternative 133, wherein the subject experiences a decrease between about a 5% to about a 100% reduction in symptoms connected to CDR Score after administration of 17α-ethynylandrost-5-ene-3β,7β,17β-triol and at least one pharmaceutically acceptable excipient.
135. The method of any one of alternatives 95 to 108, wherein administering to the subject the drug candidate or compound decreases a subject's ADAS-Cog Score.
136. The method of alternative 135, wherein the subject experiences a decrease between about a 5% to about a 100% reduction in symptoms connected to ADAS-Cog Score after administration of 17α-ethynylandrost-5-ene-3β,7β,17β-triol and at least one pharmaceutically acceptable excipient.
137. The method of any one of alternatives 95 to 108, wherein administering to the subject the drug candidate or compound decreases a subject's QDRS Score.
138. The method of alternative 137, wherein the subject experiences a decrease between about a 5% to about a 100% reduction in symptoms connected to QDRS Score after administration of 17α-ethynylandrost-5-ene-3β,7β,17β-triol and at least one pharmaceutically acceptable excipient.
139. The method of any one of alternatives 95 to 108, wherein administering to the subject the drug candidate or compound increases a subject's MMSE Score.
140. The method of alternative 139, wherein the subject experiences a decrease between about a 5% to about a 100% reduction in symptoms connected to MM SE Score or objectively normal after administration of 17α-ethynylandrost-5-ene-3β,7β,17β-triol and at least one pharmaceutically acceptable excipient.
141. The method of any one of alternatives 95 to 108, wherein administering to the subject the drug candidate or compound increases a subject's MoCA Score.
142. The method of alternative 141, wherein the subject experiences a decrease between about a 5% to about a 100% reduction in symptoms connected to MoCA Score after administration of 17α-ethynylandrost-5-ene-3β,7β,17β-triol and at least one pharmaceutically acceptable excipient.
143. The method of any one of alternatives 95 to 108, wherein administering to the subject the drug candidate or compound increases a subject's GRC Score.
144. The method of alternative 143, wherein the subject experiences a decrease between about a 5% to about a 100% reduction in symptoms connected to GRC Score or objectively normal after administration of 17α-ethynylandrost-5-ene-3β,7β,17β-triol and at least one pharmaceutically acceptable excipient.
145. The method of any one of alternatives 95 to 108, wherein administering to the subject the drug candidate or compound decreases a subject's CGIC Score.
146. The method of alternative 145, wherein the subject experiences a decrease between about a 5% to about a 100% reduction in symptoms connected to CGIC Score or objectively normal after administration of 17α-ethynylandrost-5-ene-3β,7β,17β-triol and at least one pharmaceutically acceptable excipient.
147. The method of any one of alternatives 95 to 108, wherein administering to the subject the drug candidate or compound increases a subject's ADL Score.
148. The method of alternative 147, wherein the subject experiences a decrease between about a 5% to about a 100% reduction in symptoms connected to ADL Score or objectively normal after administration of 17α-ethynylandrost-5-ene-3β,7β,17β-triol and at least one pharmaceutically acceptable excipient.
149. The method of any one of alternatives 95 to 108, wherein administering to the subject the drug candidate or compound decreases a subject's PDQ9 Score.
150. The method of alternative 149, wherein the subject experiences a decrease between about a 5% to about a 100% reduction in symptoms connected to PDQ9 Score or objectively normal after administration of 17α-ethynylandrost-5-ene-3β,7β,17β-triol and at least one pharmaceutically acceptable excipient.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1

An investigation study in early probable Alzheimer's disease was performed.

In this study, 23 mild cognitive impaired (“MCI”) and Alzheimer's disease (“AD”) subjects were enrolled based on Clinical Dementia Rating (“CDR”) from their Quick Dementia Rating System (“QDRS”) scale. Advanced MRI imaging (ASL, BOLD, MRS, task-based fMRI) was also performed as well as cognition and memory tests were performed (Cog12, MM SE, QDRS, MoCA). Biomarkers of the subjects were also taken (csf p-Tau, A beta, and plasma TNF) and episome analyses were performed. Finally, a biological clock analysis was performed (DNA methylation profiling).

It was observed for in vitro samples receiving 17α-ethynylandrost-5-ene-3β,7β,17β-triol lead to decreased activation of inflammatory p-IKK beta, p-ERK, p-P38 and p-JNK (Tau phosphorylation) which resulted in decreased p-Tau.

Under normal circumstances, insulin binds to the insulin receptor, initiating tyrosine phosphorylation of IR S1/2, leading to activation of Pi3K, A kt and inhibition of GSK 3beta. Under inflammatory conditions, activation of the inflammatory kinases p-IKK beta and p-JNK leads to serine phosphorylation of IRS-1, inhibiting insulin signaling. Inflammation thus decreases insulin stimulated Akt inhibitory phosphorylation of GSK 3beta, thus increasing p-Tau.

Without wishing to be bound by theory, it is believed that 17α-ethynylandrost-5-ene-3β,7β,17β-triol binds to ERK 1 and ERK 2, which decreases inflammatory activation of p-IKK beta, p-JNK, p-ERK and p-P38, thereby decreasing serine IRS1 phosphorylation, restoring tyrosine IRS1 phosphorylation and insulin sensitivity. It was observed that this lead to increased Akt inhibition of GSK 3beta and p-Tau.

Age related hyperglycemia results in insulin resistance, and hyperactivation of CDK 5/p25 kinase, leading not only to increased p-Tau, but also to inhibition of GSK 3beta-induced degradation of beta catenin. This combination of effects leads to neuronal cell senescence and cell death (Chow, Herrup 2019).

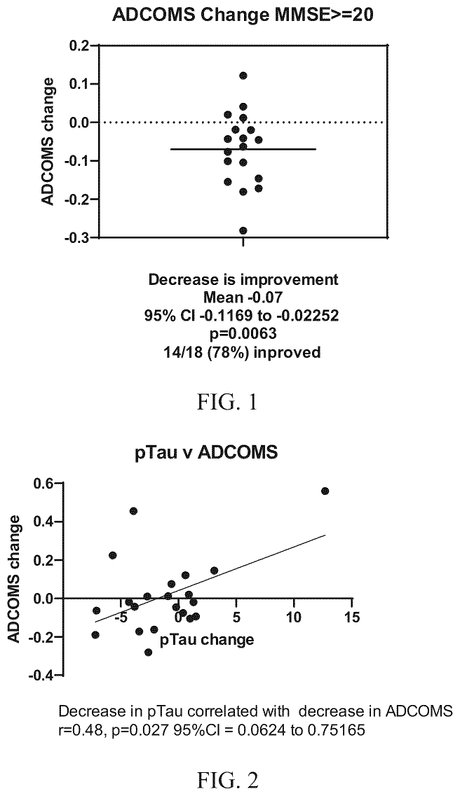

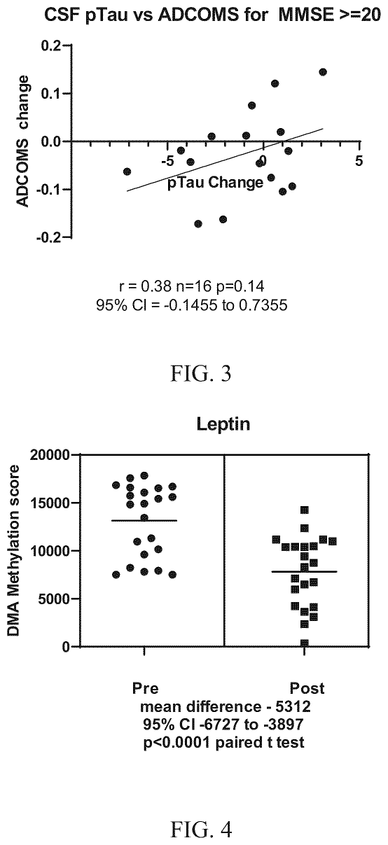

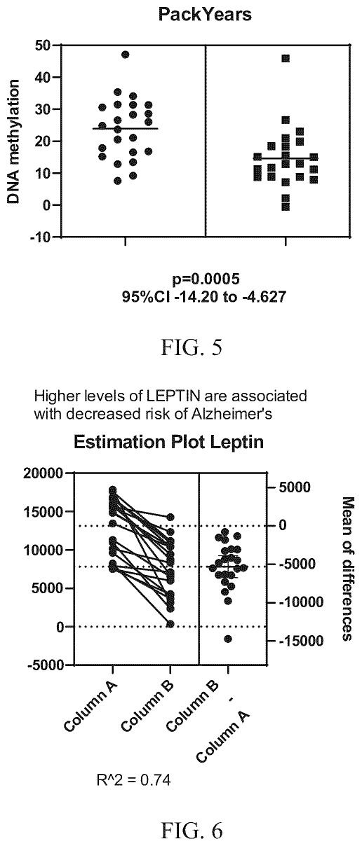

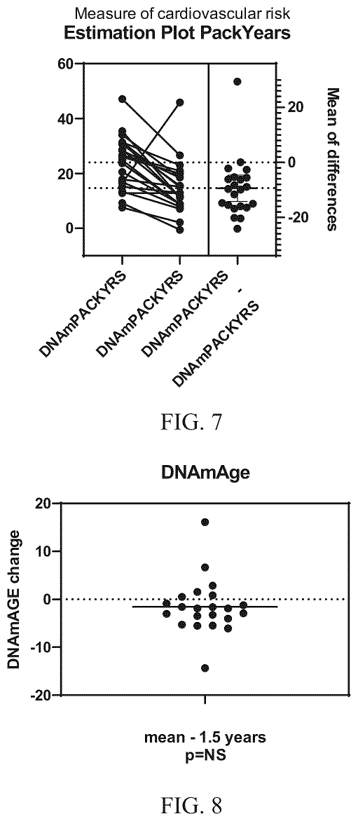

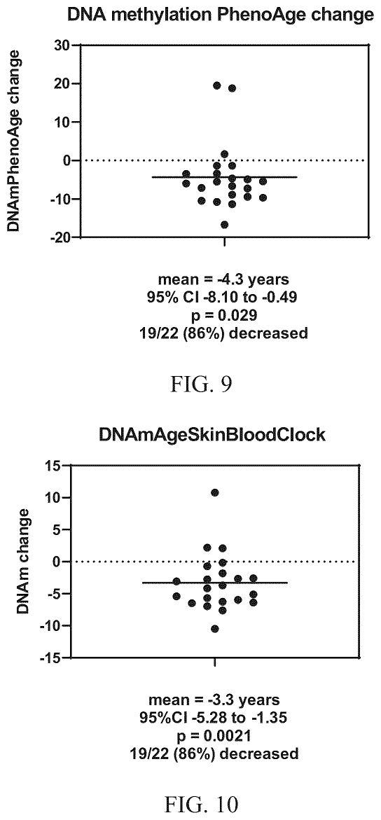

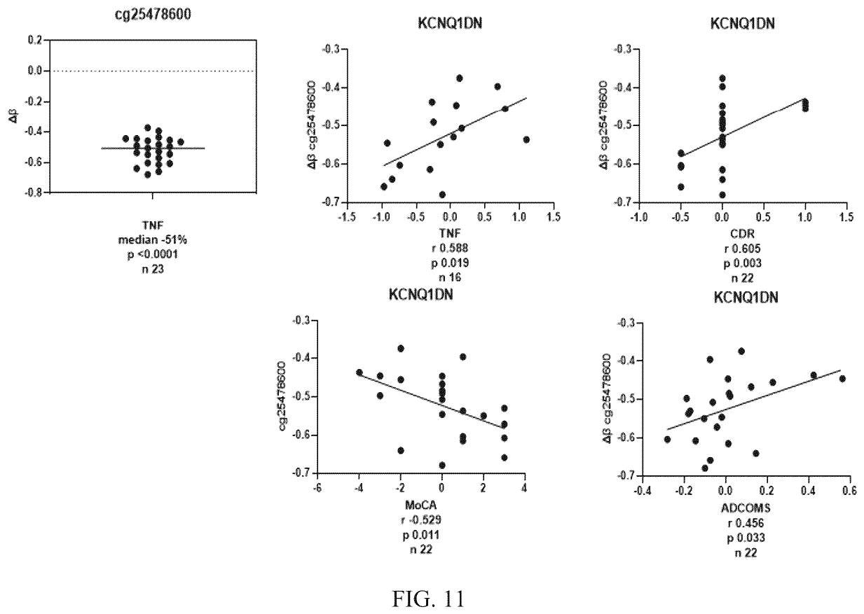

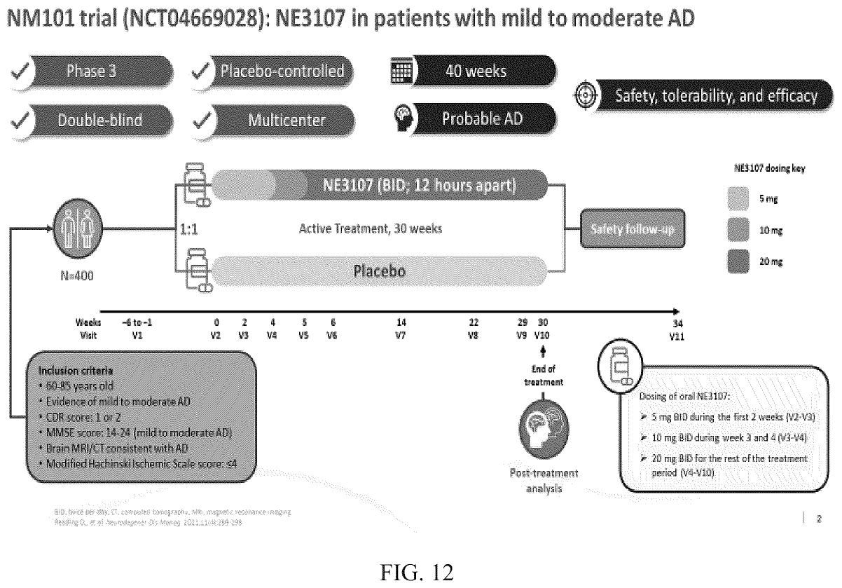

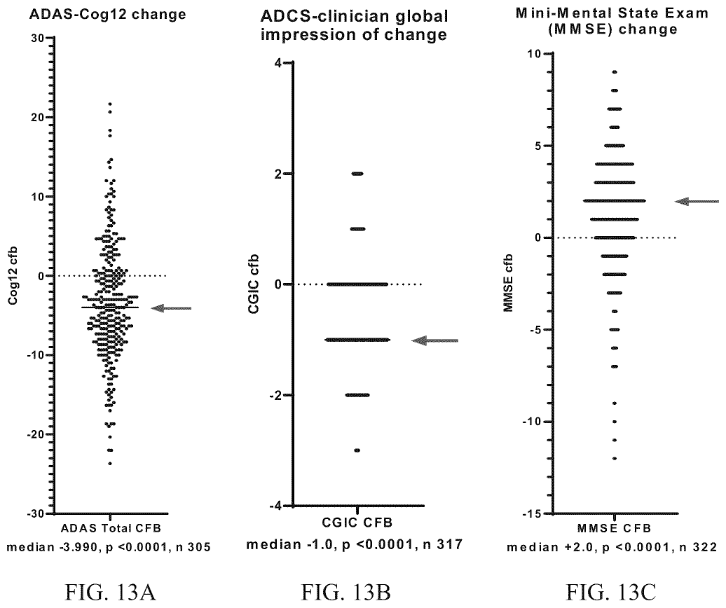

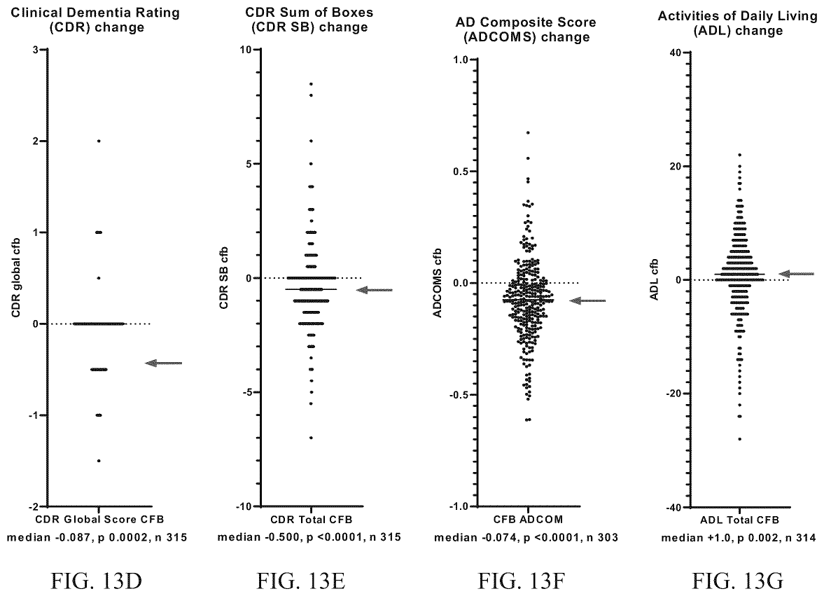

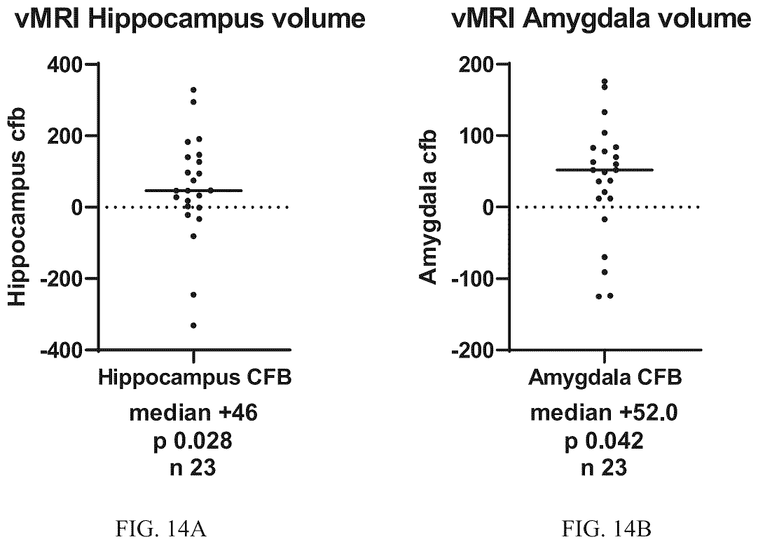

The further results of this study are illustrated in FIGS. 1-10.

### Example 2

In this study, a follow up study from Example 1 was performed.

Illumina® 850K array DNA methylation changes following 14 weeks of bezisterim treatment were sorted and the top 400 decreased CpGs (decreases >50%) were explored for correlations with changes in clinical results following treatment. Of these, 366 have CpG gene identifications. Changes for CpGs that showed Spearman correlations (p<0.05) with individual clinical changes (biomarker, cognition, function and imaging) were highly intercorrelated and were predominantly related to genes that are associated with Alzheimer's disease and related dementias.

Furthermore, many of these CpG decreases were correlated with more than one clinical change. As examples: KCNQ1DN, a potassium channel that is known to be decreased in AD brain showed a decrease in DNAm of 51% that was correlated with decreases (improvement) in TNF (inflammatory biomarker), CDR, QRDS-cognition, and an increase (improvement) in QDRS-cognition (clinical assessments). HLA-L, a protein that helps the immune system to clear amyloid plaques from the brain showed a 56% decrease in DNAm that was correlated with an improvement in subcortical grey matter thickness and frontal lobe volume (imaging). SLC26A1, decreases in this sulfate transporter is thought to contribute to cognitive decline in AD, showed a 55% decrease in DNAm That correlated with improvements in ADCOMS, QDRS (AD assessments) and CSF pTau/Aβ ratio (biomarker).

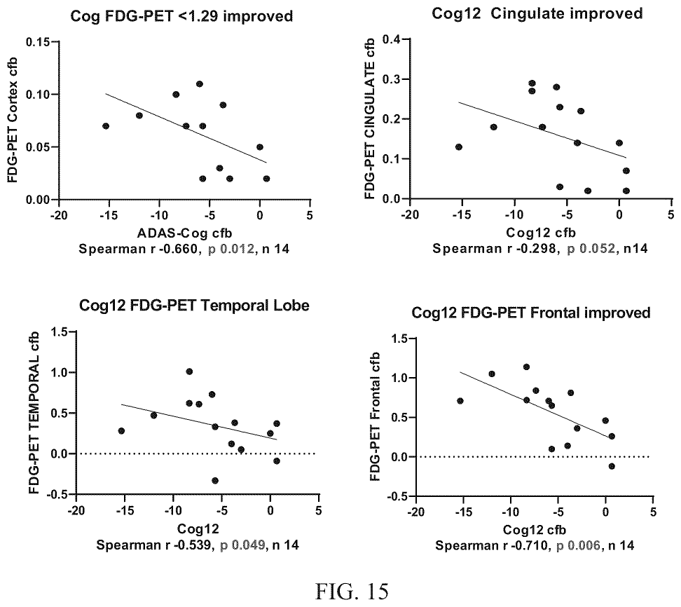

Overall significant correlations between decreases in DNAm and individual clinical changes were observed in 157 of the 366 CpGs explored, and 45 of these correlated individually with multiple clinical changes. These 157 CpGs showed individual correlations with improvements in ADAS-Cog11, ADCOMS, Ab42, CDR, CSF glucose, CSF pTau/A b, frontal lobe volume, subcortical grey matter thickness, GRC, Heart rate, MoCA, PDQ-9, precuneus glutathione, QDRS, QDRS-behavior, QDRS-cognition, Systolic BP, Tau, TNFα, and weight. The results are of this study are illustrated in FIG. 11 and Table 1.

TABLE 1

| | | DNAm | | | Clinical change | | | |
|---|---|---|---|---|---|---|---|---|
| Gene | Probe | % change | n | p | correlation | r | p | n |
| SP100 | cg02489379 | −0.54 | 23 | <0.0001 | TNFa | 0.527 | 0.039 | 16 |
| | | | | | MoCA | −0.425 | 0.048 | 22 |
| | | | | | QDRS | 0.453 | 0.034 | 22 |
| | | | | | GRC | −0.602 | 0.002 | 23 |
| KCNQ1DN | cg25478600 | −0.51 | 23 | <0.0001 | TNFa | 0.588 | 0.019 | 16 |
| | | | | | ADCOMS | 0.456 | 0.033 | 22 |
| | | | | | MoCA | −0.529 | 0.011 | 22 |
| | | | | | QDRS-Cognition | 0.503 | 0.017 | 22 |
| DBNDD2 | cg01373262 | −0.56 | 23 | <0.0001 | CDR | 0.462 | 0.031 | 22 |
| | | | | | ADCOMS | 0.522 | 0.013 | 22 |
| | | | | | Diastolic BP | −0.509 | 0.016 | 22 |
| ILKAP | cg20598555 | −0.54 | 8 | 0.0078 | CDR | 0.770 | 0.036 | 8 |
| | | | | | ADCOMS | 0.833 | 0.015 | 8 |
| | | | | | QDRS | 0.807 | 0.021 | 8 |
| CEP112 | cg01504018 | −0.53 | 23 | <0.0001 | CDR | 0.452 | 0.035 | 22 |
| | | | | | ADAS-Cog11 | 0.580 | 0.006 | 21 |
| | | | | | ADCOMS | 0.458 | 0.032 | 22 |
| | | | | | GRC | −0.517 | 0.012 | 23 |
| ANO1 | cg22082706 | −0.5212 | 23 | <0.0001 | CDR | 0.426 | 0.048 | 22 |
| | | | | | ADAS-Cog11 | 0.433 | 0.050 | 21 |
| CEP85L | cg14346555 | −0.59 | 23 | <0.0001 | ADAS-Cog11 | 0.440 | 0.046 | 21 |
| | | | | | Heart rate | 0.474 | 0.035 | 20 |
| | | | | | Grey Matter | −0.508 | 0.019 | 21 |
| ELFN2 | cg12942139 | −0.5961 | 23 | <0.0001 | ADAS-Cog11 | 0.435 | 0.049 | 21 |
| | | | | | Diastolic BP | 0.467 | 0.033 | 21 |
| | | | | | Grey matter | −0.439 | 0.047 | 21 |
| SPDYE4 | cg21610999 | −0.56 | 23 | <0.0001 | ADAS-Cog11 | 0.437 | 0.037 | 23 |
| | cg01926459 | −0.5446 | 23 | <0.0001 | ADAS-Cog11 | 0.579 | 0.006 | 21 |
| VRK2 | cg13118287 | −0.5663 | 23 | <0.0001 | ADAS-Cog11 | 0.511 | 0.018 | 21 |
| | | | | | GRC | −0.428 | 0.042 | 23 |
| HLA-L | cg14183206 | −0.5554 | 23 | <0.0001 | ADAS-Cog11 | 0.444 | 0.044 | 21 |
| | | | | | Frontal lobe | −0.497 | 0.031 | 19 |
| ZNF211 | cg25477163 | −0.53 | 23 | <0.0001 | ADAS-Cog11 | 0.564 | 0.008 | 21 |
| | | | | | Frontal lobe | −0.500 | 0.029 | 19 |
| | | | | | Grey matter | −0.449 | 0.041 | 21 |
| SDHAP3 | cg12653133 | −0.540 | 23 | <0.0001 | ADAS-Cog11 | 0.449 | 0.041 | 21 |
| | | | | | Grey matter | −0.471 | 0.031 | 21 |
| NR3C1 | cg19645279 | −0.539 | 23 | <0.0001 | ADAS-Cog11 | 0.459 | 0.037 | 21 |
| | | | | | Systolic BP | 0.470 | 0.037 | 20 |
| HLA-L | cg14183206 | −0.56 | 23 | <0.0001 | ADAS-Cog11 | 0.464 | 0.026 | 23 |
| | | | | | Grey matter | −0.559 | 0.010 | 20 |
| | | | | | Frontal lobe | −0.497 | 0.031 | 19 |
| TPP2 | cg23714043 | −0.566 | 23 | <0.0001 | ADCOMS | 0.572 | 0.005 | 22 |
| | | | | | QDRS | 0.509 | 0.016 | 22 |
| SLC26A1 | cg23958868 | −0.573 | 23 | <0.0001 | ADCOMS | 0.546 | 0.009 | 22 |
| | | | | | QDRS | 0.530 | 0.011 | 22 |
| | | | | | CSF pTau/Ab | 0.579 | 0.009 | 19 |
| SLC37A1 | cg11453546 | −0.546 | 23 | <0.0001 | ADCOMS | 0.463 | 0.030 | 22 |
| | | | | | Weight | 0.463 | 0.040 | 20 |
| CAB39L | cg00467160 | −0.544 | 23 | <0.0001 | ADCOMS | 0.590 | 0.004 | 22 |
| | | | | | QDRS | 0.435 | 0.043 | 22 |
| | | | | | GRC | −0.627 | 0.001 | 23 |
| | | | | | Diastolic BP | 0.462 | 0.035 | 21 |
| NPHP4 | cg15324288 | −0.528 | 23 | <0.0001 | ADCOMS | 0.592 | 0.004 | 22 |
| | | | | | QDRS | 0.653 | 0.001 | 22 |
| PATE4 | cg20673919 | −0.537 | 23 | <0.0001 | MMSE | −0.438 | 0.042 | 22 |
| | | | | | Amyloid b 42 | 0.611 | 0.004 | 20 |
| ARHGEF12 | cg10493270 | −0.534 | 23 | <0.0001 | MMSE | −0.477 | 0.025 | 22 |
| | | | | | Hippocampal Vol | −0.471 | 0.042 | 19 |
| CELA1 | cg11525109 | −0.559 | 23 | <0.0001 | MoCA | −0.488 | 0.021 | 22 |
| | | | | | QDRS | 0.600 | 0.003 | 22 |
| OR10G7 | cg18910882 | −0.525 | 23 | <0.0001 | QDRS behavior | 0.525 | 0.012 | 22 |
| | | | | | GRC | −0.563 | 0.005 | 23 |
| PFN2 | cg12525751 | −0.545 | 15 | <0.0001 | PDQ-9 | 0.653 | 0.013 | 14 |
| | | | | | CSF glucose | 0.457 | 0.043 | 20 |
| | | | | | Grey matter | −0.475 | 0.029 | 21 |
| WDR59 | cg00519320 | −0.514 | 23 | <0.0001 | PDQ-9 | 0.450 | 0.041 | 21 |
| | | | | | Precuneus GLTH | −0.474 | 0.040 | 19 |
| SGK | cg04463155 | −0.523 | 23 | <0.0001 | CSF glucose | 0.569 | 0.009 | 20 |
| | | | | | Weight | −0.561 | 0.010 | 20 |
| ANXA3 | cg19631365 | −0.570 | 23 | <0.0001 | CSF pTau/Ab | 0.476 | 0.040 | 19 |
| | | | | | Precuneus GLTH | −0.535 | 0.018 | 19 |
| SVIL-AS1 | cg26616244 | −0.529 | 23 | <0.0001 | CSF pTau/Ab | 0.747 | 0.007 | 12 |
| | | | | | Systolic BP | 0.492 | 0.028 | 20 |
| PPHLN1 | cg04795850 | −0.537 | 23 | <0.0001 | CSF pTau/Ab | 0.609 | 0.006 | 19 |
| | | | | | Weight | 0.474 | 0.035 | 20 |
| AP000442.1 | cg08876558 | −0.573 | 23 | <0.0001 | CSF pTau/Ab | 0.472 | 0.041 | 19 |
| | | | | | Frontal lobe | −0.500 | 0.029 | 19 |
| FA | cg24402990 | −0.515 | 23 | <0.0001 | CSF pTau/Ab | 0.524 | 0.021 | 19 |
| | | | | | Frontal lobe | −0.558 | 0.013 | 19 |
| MNAT1 | cg22431028 | −0.544 | 7 | 0.0156 | CSF pTau/Ab | 0.975 | 0.033 | 5 |
| | | | | | Grey matter | −0.857 | 0.024 | 7 |
| KIAA0319L | cg07939646 | −0.575 | 23 | <0.0001 | Amyloid b 42 | 0.445 | 0.049 | 20 |
| | | | | | Tau | 0.592 | 0.006 | 20 |
| KIAA1456 | cg11059934 | −0.558 | 23 | <0.0001 | Amyloid b 42 | 0.459 | 0.042 | 20 |
| | | | | | Diastolic BP | 0.893 | 0.012 | 7 |
| ZNF509 | cg10735211 | −0.575 | 8 | 0.0078 | Tau | 0.775 | 0.049 | 7 |
| | | | | | Weight | 0.802 | 0.048 | 7 |
| DLEU2L | cg02449698 | −0.603 | 15 | <0.0001 | Grey matter | −0.594 | 0.046 | 12 |
| | | | | | Precuneus GLTH | −0.587 | 0.049 | 12 |
| | | | | | Frontal lobe | −0.699 | 0.014 | 12 |
| | cg18803079 | −0.62 | 15 | <0.0001 | Heart rate | 0.604 | 0.032 | 13 |
| | | | | | Frontal lobe | −0.615 | 0.037 | 12 |
| | | | | | Grey matter | −0.600 | 0.026 | 14 |
| | cg10302336 | −0.567 | 23 | <0.0001 | Heart rate | 0.604 | 0.005 | 20 |
| | | | | | Grey matter | −0.446 | 0.043 | 21 |
| | cg17236673 | −0.583 | 23 | <0.0001 | Diastolic BP | 0.642 | 0.002 | 21 |
| ABL2 | cg19476594 | −0.580 | 23 | <0.0001 | Grey matter | −0.509 | 0.026 | 19 |
| | | | | | Precuneus GLTH | −0.513 | 0.025 | 19 |
| | | | | | Frontal lobe | −0.560 | 0.013 | 19 |
| | | | | | Diastolic BP | 0.503 | 0.020 | 21 |
| TMEM245 | cg17285822 | −0.592 | 23 | <0.0001 | Precuneus GLTH | −0.723 | 0.001 | 19 |
| | | | | | Frontal lobe | −0.504 | 0.028 | 19 |
| SRSF4 | cg01993027 | −0.567 | 23 | <0.0001 | Grey matter | −0.479 | 0.032 | 20 |
| | | | | | Precuneus GLTH | −0.489 | 0.034 | 19 |
| DAP | cg03278573 | −0.564 | 23 | <0.0001 | Precuneus GLTH | −0.570 | 0.011 | 19 |
| | | | | | Frontal lobe | −0.611 | 0.006 | 19 |
| LRRC69 | cg09339848 | −0.536 | 23 | <0.0001 | Precuneus GLTH | −0.542 | 0.017 | 19 |
| | | | | | Grey matter | −0.442 | 0.045 | 21 |
| GRAMD1C | cg07588614 | −0.531 | 15 | <0.0001 | Precuneus GLTH | −0.608 | 0.040 | 12 |
| | | | | | Systolic BP | 0.649 | 0.019 | 13 |
| FABP5P1 | cg14341057 | −0.58 | 23 | <0.0001 | Grey matter | −0.573 | 0.008 | 20 |
| | | | | | Frontal lobe | −0.511 | 0.026 | 19 |
| MCM10 | cg01490296 | −0.572 | 23 | <0.0001 | Heart rate | 0.483 | 0.031 | 20 |
| | | | | | Weight | −0.524 | 0.018 | 20 |
| ANP32E | cg19316489 | −0.580 | 23 | <0.0001 | Precuneus GLTH | −0.501 | 0.029 | 19 |
| | | | | | Frontal lobe | −0.521 | 0.022 | 19 |
| ZNF268 | cg19316489 | −0.58 | 23 | <0.0001 | Precuneus GLTH | −0.501 | 0.029 | 19 |
| | | | | | Frontal lobe | −0.521 | 0.022 | 19 |
| ESPN | cg04871364 | −0.567 | 23 | <0.0001 | Precuneus GLTH | −0.471 | 0.042 | 19 |
| | | | | | Frontal lobe | −0.590 | 0.008 | 19 |
| DHFR | cg04272309 | −0.541 | 23 | <0.0001 | Precuneus GLTH | −0.612 | 0.005 | 19 |
| | | | | | Frontal lobe | −0.583 | 0.009 | 19 |
| | | | | | Grey matter | −0.531 | 0.013 | 21 |
| U6 | cg25493973 | −0.521 | 23 | <0.0001 | Precuneus GLTH | −0.534 | 0.018 | 19 |
| | | | | | Frontal lobe | −0.518 | 0.023 | 19 |
| MTUS1 | cg16074739 | −0.589 | 23 | <0.0001 | Systolic BP | 0.478 | 0.033 | 20 |
| | | | | | Frontal lobe | −0.665 | 0.002 | 19 |
| ATP1B3 | cg26840922 | −0.550 | 23 | <0.0001 | Systolic BP | 0.499 | 0.025 | 20 |
| | | | | | Frontal lobe | −0.604 | 0.006 | 19 |
| RBPJ | cg21843114 | −0.550 | 23 | <0.0001 | Diastolic BP | 0.465 | 0.034 | 21 |
| | | | | | Frontal lobe | −0.688 | 0.001 | 19 |
| SLC39A9 | cg04509559 | −0.603 | 15 | <0.0001 | Heart rate | 0.593 | 0.036 | 13 |
| | | | | | Grey matter | −0.648 | 0.014 | 14 |
| TRMT1 | cg16880783 | −0.553 | 23 | <0.0001 | Heart rate | 0.455 | 0.044 | 20 |
| | | | | | Grey matter | −0.509 | 0.018 | 21 |
| PDZRN3 | cg27133230 | −0.559 | 23 | <0.0001 | Frontal lobe | −0.504 | 0.028 | 19 |
| | cg20768342 | −0.575 | 23 | <0.0001 | Frontal lobe | −0.495 | 0.031 | 19 |
| | | | | | Grey matter | −0.452 | 0.040 | 21 |

### Example 2

In this example, the clinical outcomes from a Phase 3, randomized, placebo-controlled trial of NE3107 (17α-ethynylandrost-5-ene-3β,7β,17β-triol) in subjects with mild to moderate probable Alzheimer's disease.

### Background

Recently, the roles of inflammation and insulin resistance in neurodegeneration have become better appreciated. NE3107, an oral small molecule, blood-brain permeable anti-inflammatory insulin sensitizer that binds extracellular signal-regulated kinase, has been shown to selectively inhibit inflammation-driven ERK- and NF-κB-stimulated inflammatory mediators, including TNF-α, without inhibiting their homeostatic functions. We describe the rationale and design of NM 101, the first randomized, multicenter Phase III clinical study to examine the safety and efficacy of 30-week treatment with NE3107 versus placebo in elderly adults with mild-to-moderate Alzheimer's disease. Patients (316) will be randomized in a 1:1 ratio. The co-primary end points measure cognitive function (ADAS Cog12), and functional and behavioral characteristics (ADCS CGIC). Trial registration number: NCT04669028 (Clinicaltrials.gov).

### Trial Design

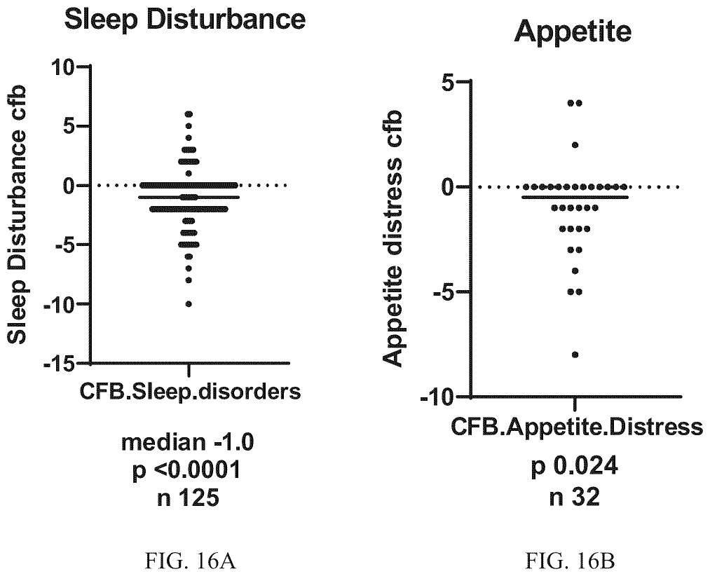

The trial design is illustrated in FIG. 12.

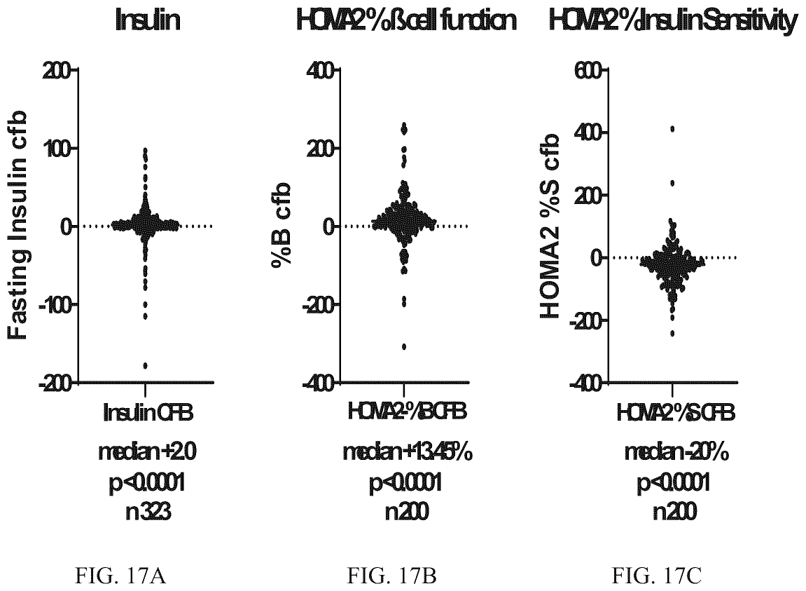

During the Phase 3 trials, the median improvement in various blinded assessments were measured. The results are described in Table 2 and FIGS. 13A-13G.

TABLE 2

| Spearman r* | CGIC | MMSE | CDR | CDR SB | ADCOMS | ADL |
|---|---|---|---|---|---|---|
| Cog12 | +0.24 | +0.46 | +0.23 | +0.23 | −0.24 | −0.40 |
| CGIC | | −0.50 | | | +0.46 | −0.40 |
| MMSE | | | −0.38 | −0.42 | −0.63 | +0.34 |
| CDR SB | | | | | +0.79 | −0.21 |
| ADCOMS | | | | | | −0.23 |

Similar distributions were observed for A POE 4+/−, mild/moderate Alzheimer's disease, male/female, and older/younger participants.

Subjects metabolic and correlation changes were analyzed. Many of the genes associated with LOAD (late onset Alzheimer's disease) are related to cholesterol metabolism, which is decreased in AD neuron membranes. Insulin and glycemic controls are known to be involved in neurodegeneration. Increased insulin and HOMA2-% B cell function, and decreased HOMA2% insulin sensitivity resulted in no cases of hypoglycemia. Increased mean amplitude of glycemic excursion (MAGE from CGM) increased risk of Alzheimer's disease progression. Leptin is an anti-inflammatory, neuroprotective and is decreased in AD. Leptin DNAm was observed to increase. The results are described in Tables 4 and 5.

TABLE 4

| | Cholesterol | Insulin | HOMA2-% B | HOMA2-% S | MAGE | Leptin |
|---|---|---|---|---|---|---|
| Change | +3.0 mg/dL | +1.9 μlU/mL,* | +13.5%, * | −21.2%, * | −0.57 mg/dL | |
| from | | | | | | |
| Baseline | | | | | | |
| V10 median | | | | | | 29.2 ng/mL |

* P < 0.0001

TABLE 5

| Spearman r | CGIC | ADL | Cog12 | MMSE |
|---|---|---|---|---|
| Cholesterol | +0.170** | +0.130* | −0.164** | |
| MAGE | +0.253* | | | |
| Leptin# | | | −0.264* | 0.344* |

*p < 0.05,
**p < 0.005,
***p < 0.0001,
#excludes obese subjects

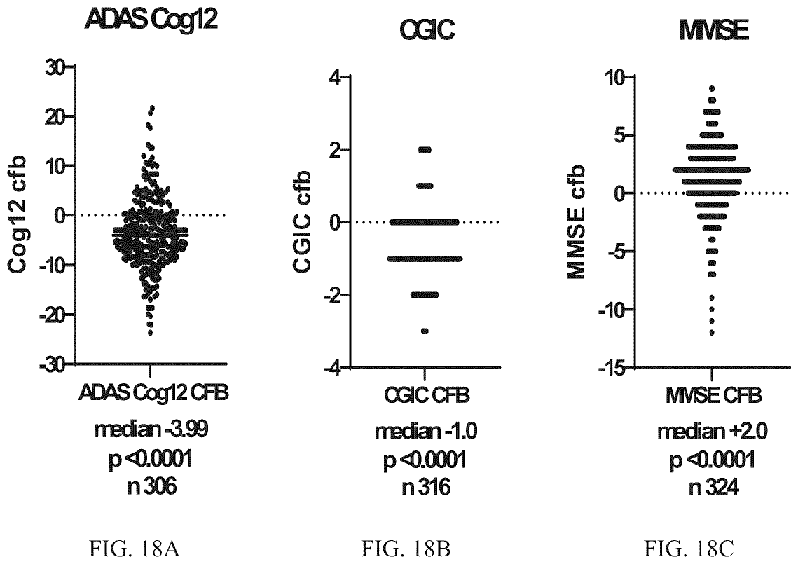

Subjects were analyzed using imaging sub-studies vMRI on 23 subjects. It was observed that the volume was increased in the hippocampus and amygdala. In addition, it was observed a decreased CGIC was correlated with decreased hippocampi volume and increased MCP-1 was correlated with increased whole cortex. The results are described in FIGS. 14A-14B and Table 6.

TABLE 6

| Spearman r | Whole Cortex | Hippocampus |
|---|---|---|
| CGIC | −0.458** | |
| MCP-1 | | +0.713** |

**p < 0.05

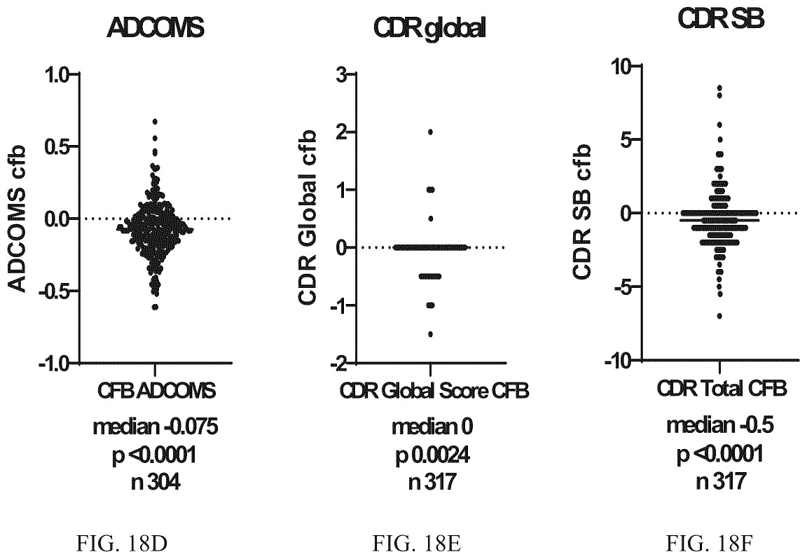

Subjects were analyzed using a fluorodeoxyglucose (FDG)-positron emission tomography (PET) imaging scans. FDG-PET standardized uptake value ratios (“suvr”) were increased in 14/24 subjects with a baseline whole cortex suvr <1.29. This data correlates with Cog12 and ADL with suvr improvements. Cholesterol increases trended with increased Cingulate suvr. The results are described in Table 7 and FIG. 15.

TABLE 7

| Spearman r | Cortex | Cingulate | Temporal | Parietal | Frontal |
|---|---|---|---|---|---|
| PET (+) | −0.660** | −0.534* | −0.540 | −0.197 | −0.710*** |
| Cog12 | | | | | |
| PET (−) | −0.298 | −0.298 | −0.115 | −0.165 | −0.114 |
| Cog12 | | | | | |
| PET (+) | +0.546** | +0.623** | +0.523** | −0.197 | +0.358 |
| ADL | | | | | |
| PET (−) ADL | −0.146 | −0.298 | −0.115 | −0.164 | −0.114 |
| Cholesterol | | +0.424* | | | |

*p < 0.1,
**p < 0.05,
***p < 0.01

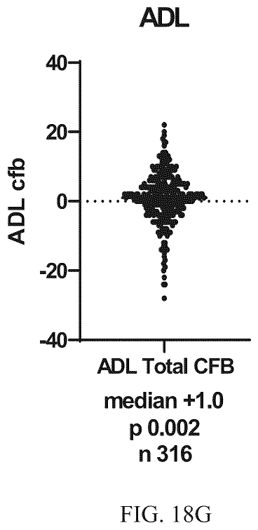

Subjects were analyzed for neuropsychiatric inventory. It was observed that overall improvement was seen in sleep (−1.0, p<0.0001) and appetite (−1.0, p<0.023). An improvement in appetite was also correlated with decreased Cog12, CDR SB and ADCOMS, with increased Alzheimer's disease. A decrease in anxiety was correlated with an increase ADL and decreased CGIC. Sleep improvement was correlated with a decrease in CGIC and decreased in TNFα. The results are described in Table 8 and FIGS. 16A-16B.

TABLE 8

| Spearman r | Cog12 | CG12 | CDR SB | ADCOMS | ADL | TNFα |
|---|---|---|---|---|---|---|
| Appetite | +0.54*** | | +0.36** | +0.36* | −0.65**** | |
| Anxiety | | +0.15* | | | −0.14* | |
| Sleep | | +0.22** | | | | +0.24* |

*p < 0.10, **p < 0.05, ***p < 0.01, ****p < 0.001

The study overall had a very low rate of adverse effects (AEs) reported and only 10 subjects discontinued due to a reported A E (2.3%). Of the 439 subjects enrolled in the study, 156 experienced 1 or more AEs (35.5%). There were only 43 (9.8%) related AEs with the majority 42 ((0.6%) categorized as non-serious AEs by the principal investigator. The study also had very low rate of serious AEs reported. There were 12 (2.7%) reported for the duration of the study. Only 1 of the serious AEs reported resulted in death. As this is a blinded analysis it is not known if the subjects was on IP or placebo.

Additional metrics were measured during the Phase 3 studies as represented in FIGS. 16A-33.

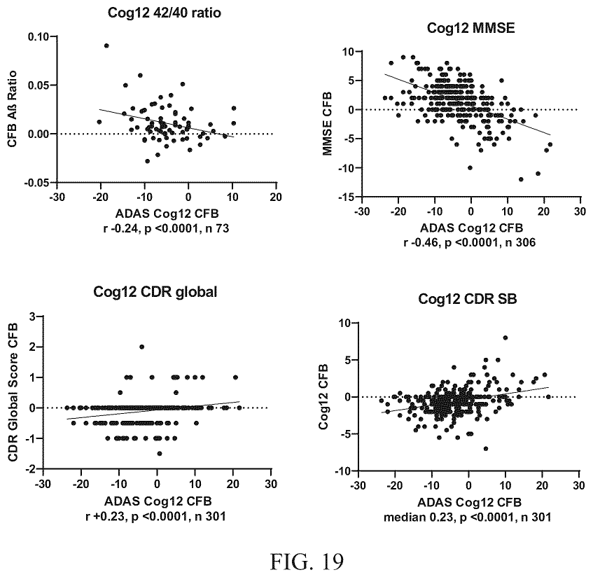

FIGS. 17A-17C illustrates graphs representing increased fasting insulin and HOMA2-% B with decreased HOMA2-% S w/o hypoglycemia. Table 10 provides additional information regarding the fasting insulin and HOMA2-% B with decreased HOMA2-% S w/o hypoglycemia.

TABLE 9

| Increased Fasting Insulin | Hypoglycemia |
|---|---|
| Yes | 0 |
| No | 2 |

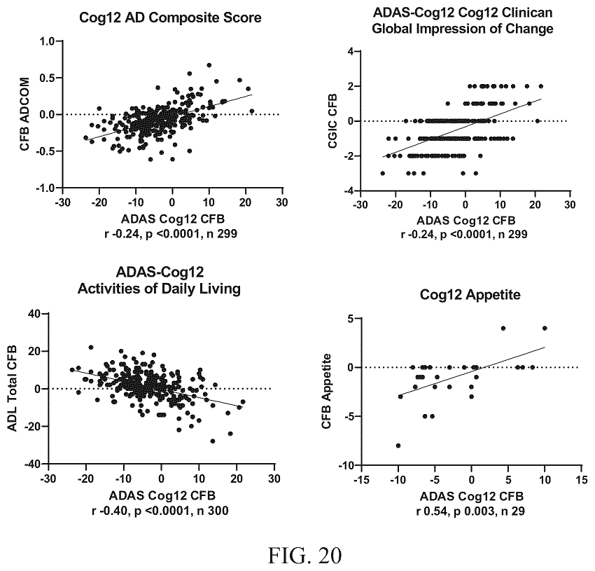

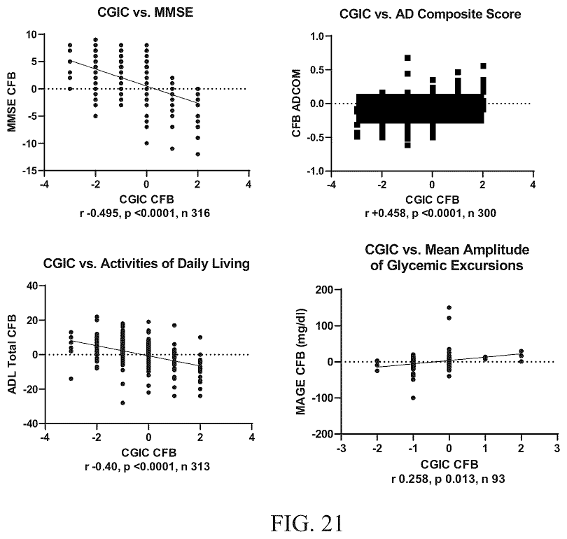

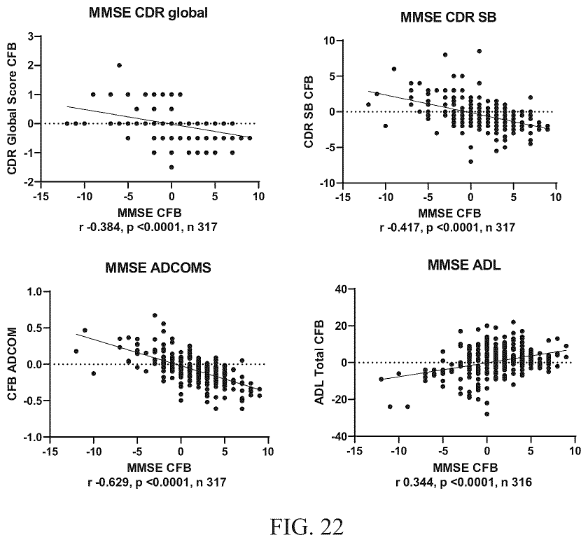

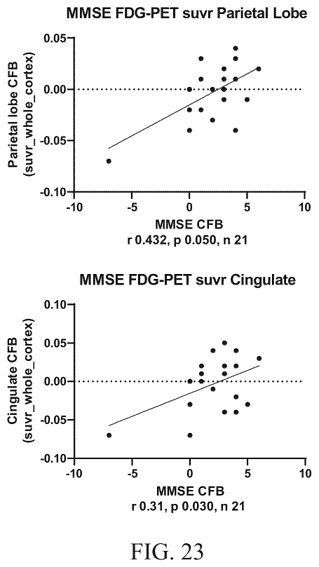

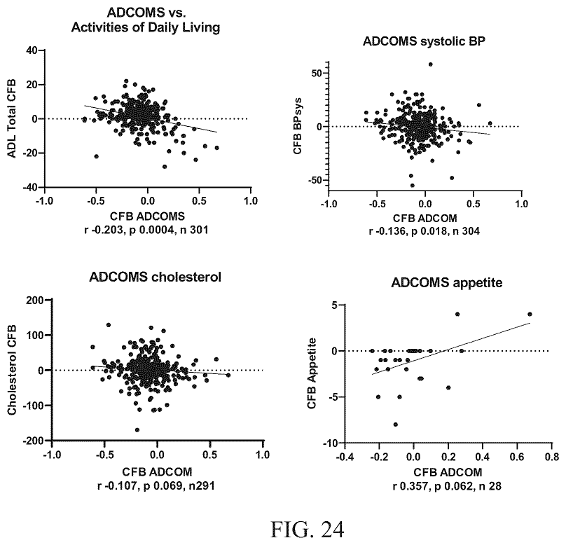

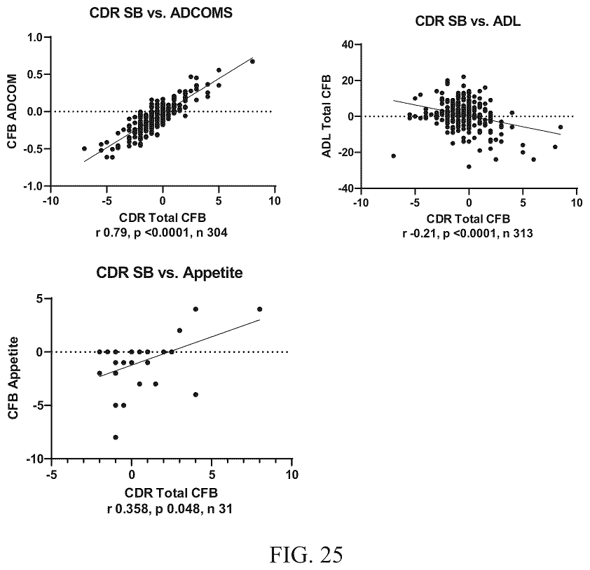

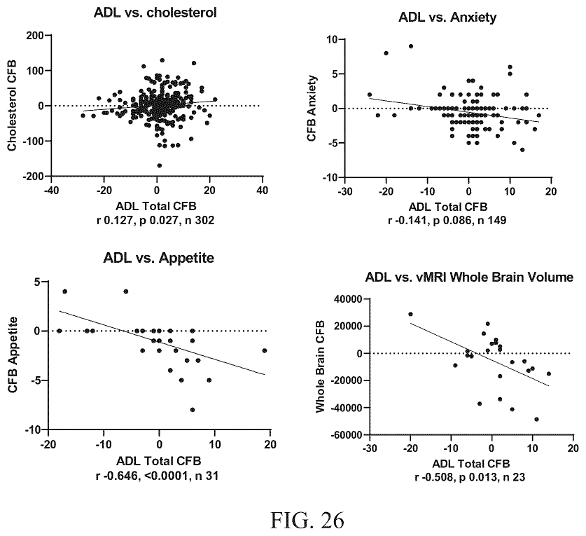

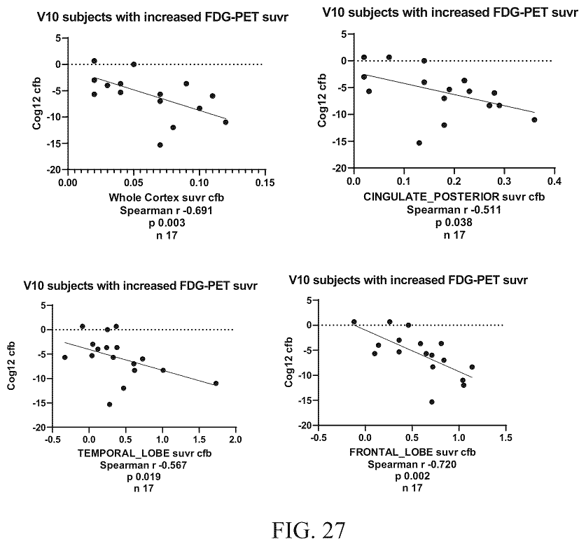

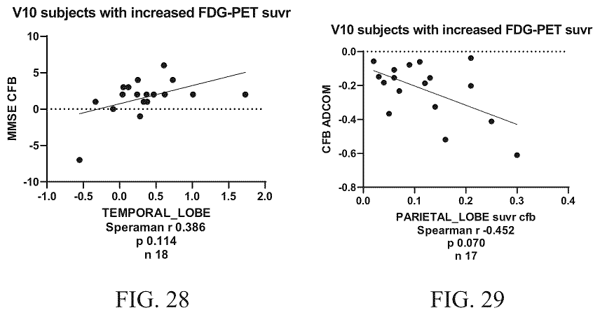

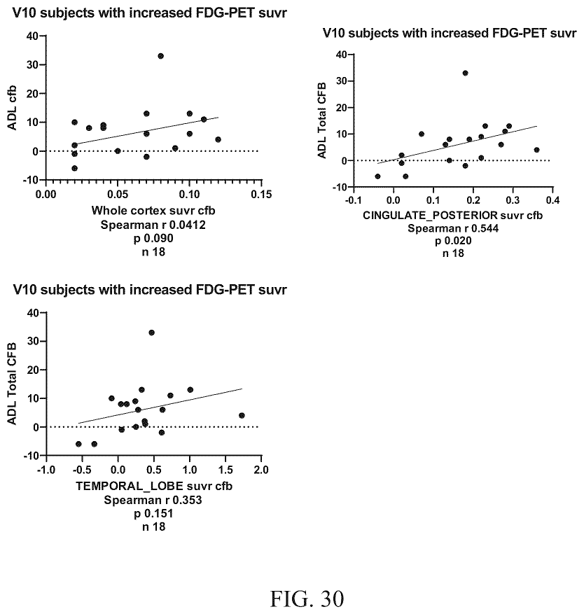

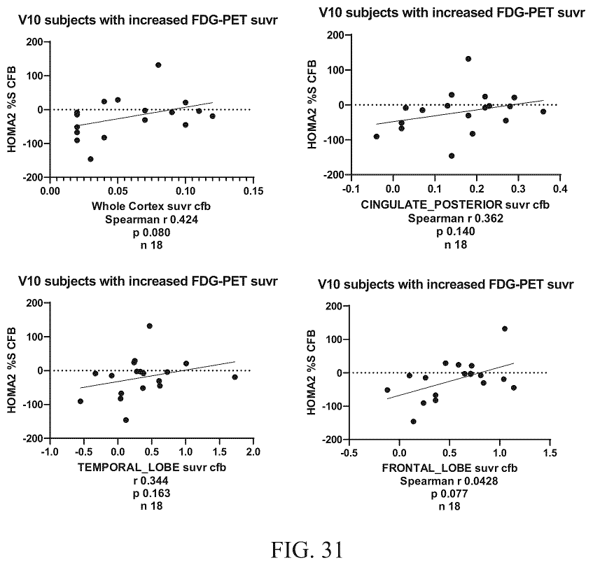

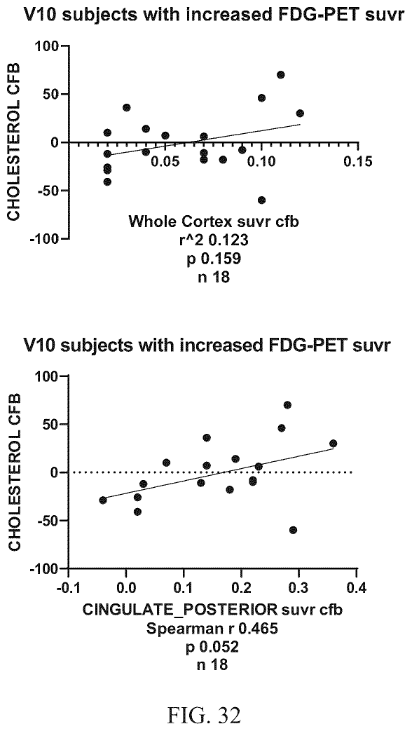

FIGS. 18A-18G illustrate graphs representing placebo effects in various assessments. Medians showing improvement indicate that at least some placebo subjects showed improvement in neurocognitive and functional assessments. Similar findings in vMRI, NPI, HOMA, and APS suggest this is not just related to assessment inflation at baseline. FIGS. 19-20 illustrate graphs representing A DAS-Cog12 spearman correlations. FIGS. 21-22 illustrate graphs representing clinician rating of global change spearman correlations. FIGS. 23-24 illustrate graphs representing mini-mental state exam spearman correlations. FIG. 25 illustrate graphs representing ADCOMS spearman correlations. FIG. 26 illustrate graphs representing CDR sum of boxes spearman correlations. FIG. 27 illustrate graphs representing activities of daily living spearman correlations. A subset of subjects participated in FDG-PET analysis, and an increase in glucose uptake was observed in about half of the FDG-PET subjects. FIG. 28 illustrate graphs representing improvement in A DAS-Cog12 correlated with increased FDG-PET suvr. FIG. 29 illustrate a graph representing an improvement in MMSE correlated with increased FDG-PET suvr. FIG. 30 illustrate graphs representing an improvement in ADL correlated with increased FDG-PET suvr. FIG. 31 illustrate graphs representing an improvement in HOMA2 insulin sensitivity correlated with increased FDG-PET suvr. FIG. 32 illustrate graphs representing an improvement in cholesterol correlated with increased FDG-PET suvr.

Improvements in the blinded data for cognitive and accepted Alzheimer's disease biomarkers (Aβ and FDG-PET) suggests NE3107 is active in subjects with mild/moderate AD. The blinded analysis were also consistent with the hypothesis on NE3107's anti-inflammatory and insulin sensitizing activity in Alzheimer's disease.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

What is claimed is:

1. A method to treat, reduce, or ameliorate a disease or condition associated with biological clocks in a subject in need thereof, the method comprising:
administering to the subject 17α-ethynylandrost-5-ene-3β,7β,17β-triol.

2. The method of claim 1, wherein the disease or condition associated with biological clocks in the subject in need thereof is based on modulation of DNA methylation of genes associated with biological clocks.

3. The method of claim 1 or 2, wherein the disease or condition associated with a biological clock in the subject in need thereof is associated with genes or genomic regions hypermethylated with age.

4. The method of any one of claims 1 to 3, wherein the disease or condition associated with a biological clock in the subject in need thereof is associated with genes or genomic regions hypomethylated with age.

5. The method of any one of claims 1 to 4, wherein the disease or condition associated with a biological clock in a subject in need thereof is associated with Tau phosphorylation.

6. The method of any one of claims 1 to 5, wherein the disease or condition associated with a biological clock in a subject in need thereof is associated with hyperglycemia.

7. The method of any one of claims 1 to 6, wherein the disease or condition associated with a biological clock in a subject in need thereof is associated with insulin resistance.

8. The method of any one of claims 1 to 7, wherein the disease or condition associate with biological clock is mild cognitive impairment or late onset Alzheimer's disease.

9. The method of any one of claims 1 to 8, wherein administering to the subject 17α-ethynylandrost-5-ene-3β,7β,17β-triol and at least one pharmaceutically acceptable excipient decreases a subject's Alzheimer's Disease Composite Score.

10. The method of any one of claims 1 to 8, wherein administering to the subject 17α-ethynylandrost-5-ene-3β,7β,17β-triol and at least one pharmaceutically acceptable excipient decreases leptin in the subject.

11. The method of claim 10, wherein the subject experiences an increase between about a 5% to about a 100% in conditions or symptoms connected to the leptin after administration of 17α-ethynylandrost-5-ene-3β,7β,17β-triol and at least one pharmaceutically acceptable excipient.

12. The method of any one of claims 1 to 8, wherein administering to the subject 17α-ethynylandrost-5-ene-3β,7β,17β-triol and at least one pharmaceutically acceptable excipient decreases DNA methylation in the subject.

13. The method of claim 12, wherein the subject experiences a decrease between about a 5% to about a 100% reduction in conditions or symptoms connected to DNA methylation after administration of 17α-ethynylandrost-5-ene-3β,7β,17β-triol and at least one pharmaceutically acceptable excipient.

14. The method of any one of claims 1 to 8, wherein administering to the subject 17α-ethynylandrost-5-ene-3β,7β,17β-triol and at least one pharmaceutically acceptable excipient decreases cardiovascular risk in the subject.

15. The method of claim 14, wherein the subject experiences a decrease between about a 5% to about a 100% reduction in conditions or symptoms connected to the cardiovascular risk after administration of 17α-ethynylandrost-5-ene-3β,7β,17β-triol and at least one pharmaceutically acceptable excipient.

16. The method of any one of claims 1 to 8, wherein administering to the subject 17α-ethynylandrost-5-ene-3β,7β,17β-triol and at least one pharmaceutically acceptable excipient decreases DNA methylation age in the subject.

17. The method of claim 16, wherein the subject experiences a decrease between about a 5% to about a 100% reduction in conditions or symptoms connected to DNA methylation after administration of 17α-ethynylandrost-5-ene-3β,7β,17β-triol and at least one pharmaceutically acceptable excipient.

18. The method of any one of claims 1 to 8, wherein administering to the subject 17α-ethynylandrost-5-ene-3β,7β,17β-triol and at least one pharmaceutically acceptable excipient decreases DNA methylation phenoage in the subject.

19. The method of claim 18, wherein the subject experiences a decrease between about a 5% to about a 100% reduction in conditions or symptoms connected to DNA methylation phenoage after administration of 17α-ethynylandrost-5-ene-3β,7β,17β-triol and at least one pharmaceutically acceptable excipient.

20. The method of any one of claims 1 to 8, wherein administering to the subject 17α-ethynylandrost-5-ene-3β,7β,17β-triol and at least one pharmaceutically acceptable excipient decreases DNA methylation skin blood clock in the subject.

21. The method of claim 20, wherein the subject experiences a decrease between about a 5% to about a 100% reduction in conditions or symptoms connected to the DNA methylation skin blood clock after administration of 17α-ethynylandrost-5-ene-3β,7β,17β-triol and at least one pharmaceutically acceptable excipient.

22. The method of any one of claims 1 to 8, wherein administering to the subject 17α-ethynylandrost-5-ene-3β,7β,17β-triol and at least one pharmaceutically acceptable excipient decreases a subject's CDR Score.

23. The method of claim 22, wherein the subject experiences a decrease between about a 5% to about a 100% reduction in symptoms connected to CDR Score after administration of 17α-ethynylandrost-5-ene-3β,7β,17β-triol and at least one pharmaceutically acceptable excipient.

24. The method of any one of claims 1 to 8, wherein administering to the subject 17α-ethynylandrost-5-ene-3β,7β,17β-triol and at least one pharmaceutically acceptable excipient decreases a subject's ADAS-Cog Score.

25. The method of claim 24, wherein the subject experiences a decrease between about a 5% to about a 100% reduction in symptoms connected to ADAS-Cog Score after administration of 17α-ethynylandrost-5-ene-3β,7β,17β-triol and at least one pharmaceutically acceptable excipient.

26. The method of any one of claims 1 to 8, wherein the subject experiences a decrease between about a 5% to about a 100% reduction in symptoms connected to MMSE Score after administration of 17α-ethynylandrost-5-ene-3β,7β,17β-triol and at least one pharmaceutically acceptable excipient.

27. The method of any one of claims 1 to 8, wherein administering to the subject 17α-ethynylandrost-5-ene-3β,7β,17β-triol and at least one pharmaceutically acceptable excipient decreases a subject's M oCA Score.

28. The method of any one of claims 1 to 8, wherein the subject experiences a decrease between about a 5% to about a 100% reduction in symptoms connected to ADL Score after administration of 17α-ethynylandrost-5-ene-3β,7β,17β-triol and at least one pharmaceutically acceptable excipient.

29. The method of any one of claims 1 to 28, wherein the 17α-ethynylandrost-5-ene-3β,7β,17β-triol is administered orally.

30. The method of any one of claims 1 to 28, wherein the 17α-ethynylandrost-5-ene-3β,7β,17β-triol is administered intravenously.

31. The method of any one of claims 1 to 30, wherein the subject has a waist to hip ratio greater than or equal to approximately 0.90.

32. The method of any one of claims 1 to 30, wherein the subject has a waist to hip ratio greater than or equal to approximately 0.95.

33. The method of any one of claims 1 to 32, wherein the 17α-ethynylandrost-5-ene-3β,7β,17β-triol is a solid state form of 17α-ethynylandrost-5-ene-3β,7β,17β-triol.

34. The method of claim 33, wherein the solid state form of 17-ethynylandrost-5-ene-3β,7β,17β-triol is crystalline solvate of 17α-ethynylandrost-5-ene-3β,7β,17β-triol.

35. The method of claim 34, wherein the crystalline solvate is crystalline methanolate 17α-ethynylandrost-5-ene-3β,7β,17β-triol.

36. The method of claim 35, wherein the crystalline solvate is crystalline ethanolate 17α-ethynylandrost-5-ene-3β,7β,17β-triol.

37. The method of claim 36, wherein the crystalline solvate is crystalline hydrate 17α-ethynylandrost-5-ene-3β,7β,17β-triol.

38. The method of claim 36, wherein the crystalline solvate is Form III 17α-ethynylandrost-5-ene-3β,7β,17β-triol.

39. The method of claim 36, wherein the crystalline solvate is Form IV 17α-ethynylandrost-5-ene-3β,7β,17β-triol.

40. The method of claim 36, wherein the crystalline solvate is Form V 17α-ethynylandrost-5-ene-3β,7β,17β-triol.

41. The method of claim 35, wherein the solid state form of 17α-ethynylandrost-5-ene-3β,7β,17β-triol is amorphous 17α-ethynylandrost-5-ene-3β,7β,17β-triol.

42. The method of any one of claims 1 to 41, wherein the 17α-ethynylandrost-5-ene-3β,7β,17β-triol is in a pharmaceutical composition, wherein the pharmaceutical composition contains less than about 3% by weight of impurities.

43. An in vitro screening method to identify a potential drug candidate or compound capable of treating, preventing, reducing, or ameliorating a disorder or disease, comprising:
(i) providing a sample comprising one or more cells for stimulation selected from the group consisting of a cell, tissue, blood, monocytes, microglia, macrophages, adipocytes, neuroblastoma, pheochromocytoma, and Lund human mesencephalic (LUHMES) cells;
(ii) stimulating the sample with an agonist to induce a phenotype or phenotypic reaction, wherein the phenotype or phenotypic reaction substantially corresponds to a disease or condition associated with at least one DNA methylation at a CpG site in a region of DNA;
(iii) contacting the one or more cells exhibiting the phenotype or phenotypic reaction with one or more potential drug candidate;
(iv) determining a responsive change in the phenotype or phenotypic reaction of the sample; and
(v) providing the drug candidate to a subject in need thereof to treat, reduce, prevent, or ameliorate the disease or condition associated with the DNA methylation in the subject.

44. The method of claim 43, wherein the at least one DNA methylation at the CpG site is selected from the group consisting of AC073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAMD1C, FABP5P1, MCM10, ANP32E, ZNF268, ESPN, DHFR, U6, MTUS1, ATP1B3, or a combination thereof.

45. The method of any one of claims 43 to 44, wherein the responsive change is a decrease or loss in the phenotype and the decrease or loss is indicative that the potential drug candidate is capable of preventing, reducing, or ameliorating a neurodegenerative disorder or disease.

46. The method of claim 45, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

47. The method of claim 46, wherein identifying DNA methylation changes in the subject identifies a CpGs decreased by more than 50%.

48. The method of claim 47, wherein identifying DNA methylation changes is correlated with one or more clinical changes.

49. The method of any one of claims 44 to 48, wherein the DNA methylation changes at a CpG site is selected from the group consisting of A C073869.20, SP100, KCNQ1DN, DBNDD2, CEP112, CEP85L, SPDYE4, ZNF211, NR3C1, HLA-L, TPP2, SLC26A1, SLC37A1, CAB39L, ILKAP, NPHP4, PATE4, ARHGEF12, CELA1, OR10G7, PFN2, WDR59, snoU13, ANXA3, SVIL-AS1, PPHLN1, AP000442.1, FA, KIAA0319L, ZNF509, DLEU2L, ABL2, SGK1, TMEM245, SRSF4, DAP, GRAM DIC, FABP5P1, MCM10, ANP32E, ZNF268, ESPN, DHFR, U6, MTUS1, ATP1B3, or a combination thereof.

50. The method of any one of claims 44 to 49, wherein the disease or condition associated with a biomarker associated with DNA methylation is selected from the group consisting of TNFα, GRC, CDR, MoCA, QDRS, GRC, ADCOMS, MoCA, QDRS-Cognition, ADAS-Cog11, heart rate, frontal lobe, systolic blood pressure, grey matter, weight, MMSE, hippocampal volume, behavior, PDQ-9, CSF glucose, precuneus GLTH, CSF pTau/Ab, or a combination thereof.
